(19) United States
(12) Patent Application Publication
(10) Pub. No.: US 2024/0316074 A1
(43) Pub. Date: Sep. 26, 2024

(54) METHODS OF TREATING OR PREVENTING CORONAVIRUS INFECTION WITH CANNABINOID ACIDS

(71) Applicant: Oregon State University, Corvallis, OR (US)

(72) Inventors: Richard B. van Breemen, Corvallis, OR (US); Ruth N. Muchiri, Corvallis, OR (US)

(73) Assignee: Oregon State University, Corvallis, OR (US)

(21) Appl. No.: 18/575,686

(22) PCT Filed: Jun. 30, 2022
(86) PCT No.: PCT/US2022/035708

§ 371 Date: Dec. 29, 2023

Related U.S. Application Data
(60) Provisional application No. 63/218,137, filed on Jul. 2, 2021.

(51) Int. Cl. A61K 31/00; A61P 31/14

(52) U.S. Cl. CPC A61K 31/658; A61P 31/14

(57) ABSTRACT

Described herein are methods and pharmaceutical compositions for treating coronavirus-induced disease or preventing infection by a coronavirus by administering an effective amount of a cannabinoid acid or a pharmaceutically acceptable salt, stereoisomer, or prodrug thereof, to a subject.

### CROSS-REFERENCE TO RELATED APPLICATION

This application claims the benefit of U.S. Patent Application No. 63/218,137, filed Jul. 2, 2021, expressly incorporated herein by reference in its entirety.

### STATEMENT REGARDING SEQUENCE LISTING

The sequence listing associated with this application is provided in text format in lieu of a paper copy and is hereby incorporated by reference into the specification. The name of the text file containing the sequence listing is 3014P25WO_Seq_List_20220629_ST25. The text file is 11.4 KB; was created on Jun. 29, 2022; and is being submitted via EFS-Web with the filing of the specification.

### BACKGROUND

β coronavirus infection can cause not only severe respiratory system distress and lung damage, but also can illicit an overreaction of the body's immune system. SARS-CoV-2, for example, causes an acute respiratory disease, COVID-19, which can be fatal. Although vaccines have been developed, due to their limited availability and the rate of virus mutation, SARS-CoV-2 infections are likely to continue for many years. As the pandemic continues, several SARS-CoV-2 variants have emerged that are circulating globally, including the variant B.1.1.7 (first detected in the United Kingdom) and variant B.1.351 (first detected in South Africa). These variants of concern are reported to have the capacity to escape humoral immunity elicited by natural infection or the current vaccinations. Moreover, the variants are associated with increases in infections and hospitalizations, suggesting a competitive fitness advantage over the original strain. Accordingly, additional methods treating or preventing coronaviruses, such as SARS-CoV-2 are needed.

### SUMMARY

In aspects, the present disclosure provides a method for treating a β coronavirus-induced disease in a subject in need thereof, the method comprising administering an effective amount of a cannabinoid acid or a pharmaceutically acceptable salt, stereoisomer, or prodrug thereof, to the subject.

In further aspects, the present disclosure provides a method for preventing infection by a β coronavirus in a subject in need thereof, the method comprising administering an effective amount of a cannabinoid acid or a pharmaceutically acceptable salt, stereoisomer, or prodrug thereof, to the subject.

In additional aspects, the present disclosure provides a method for identifying a ligand that binds to SARS-CoV-2 spike protein, comprising: mixing, in a well, the ligand and the SARS-CoV-2 spike protein such that the ligand binds to the SARS-CoV-2 spike protein, wherein the SARS-CoV-2 spike protein has been immobilized on a magnetic bead; capturing the ligand bound to the SARS-CoV-2 spike protein by introducing a magnet into the well; releasing the ligand; and analyzing the ligand using liquid chromatography-mass spectrometry.

In one aspect, the present disclosure provides a method for preventing infection by a β coronavirus in a subject or treating a β coronavirus-induced disease in a subject in need thereof, the method comprising administering an effective amount of a cannabinoid acid (i.e., one or more cannabinoid acids) or a pharmaceutically acceptable salt, stereoisomer, or prodrug thereof, to the subject, wherein the cannabinoid acid has formula (II):

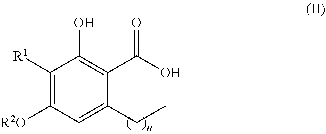

wherein
R¹ is alkenyl or alkenylcycloalkenyl;
R² is H or R¹ and R² join to form a tetrahydro- or dihydropyan ring;
n is an integer from 1 to 8,
wherein each occurrence of alkenyl and alkenylcycloalkenyl is optionally substituted with one or more substituents.

In certain of these embodiments, the method comprises administering to the subject an effective amount of a composition comprising, consisting essentially of, or consisting of a cannabinoid acid (i.e., one or more cannabinoid acids), or a pharmaceutically acceptable salt, stereoisomer, or prodrug thereof, wherein the cannabinoid acid has formula (II), and optionally further includes a pharmaceutically acceptable carrier.

In certain embodiments, the cannabinoid acid is selected from the group consisting of cannabigerolic acid (CBGA), tetrahydrocannabinolic acid (THCA-A), cannabidiolic acid (CBDA), cannabinolic acid (CBNA), pharmaceutically acceptable salts, stereoisomers, or prodrugs thereof, and mixtures thereof.

In certain embodiments, the cannabinoid acid is a combination of CBDA, or pharmaceutically acceptable salt, stereoisomer, or prodrug thereof, and CBGA, or pharmaceutically acceptable salt, stereoisomer, or prodrug thereof. In other embodiments of the above method, the cannabinoid acid is a combination of THCA-A, or pharmaceutically acceptable salt, stereoisomer, or prodrug thereof, and CBGA, or pharmaceutically acceptable salt, stereoisomer, or prodrug thereof.

In certain embodiments, the cannabinoid acid is CBDA, or pharmaceutically acceptable salt, stereoisomer, or prodrug thereof, and wherein administering an effective amount of a cannabinoid acid or a pharmaceutically acceptable salt, stereoisomer, or prodrug thereof, further comprises administering to the subject an effective amount a compound that inhibits (efflux) transport of CBDA out of cells (e.g., CBD or CBG).

In another aspect, the present disclosure provides pharmaceutical compositions that include a cannabinoid acid (i.e., one or more cannabinoid acids). In certain embodiments, the pharmaceutical composition includes a cannabinoid acid of formula (II), or pharmaceutically acceptable salt, stereoisomer, or prodrug thereof.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1A shows the spike protein of SARS-CoV-2, which consists of trimers of a protein containing an S1 subunit, an S2 subunit, and a transmembrane domain. The S1 subunit binds to human ACE2 to initiate cell entry. Recombinant S1 containing a His-tag was immobilized on magnetic microbeads for affinity selection of ligands.

FIG. 1B shows how AS-MS was used to isolate and identify natural ligands to the spike protein S1 subunit. A magnetic probe retained the microbeads containing the S1 subunit and bound ligands while unbound compounds were washed away. Ligands were released using organic solvent and then analyzed using UHPLC-MS.

FIG. 1C shows that during AS-MS, the SBP-1 peptide bound to immobilized S1 (equivalent to 0.17 μM) (positive control) but not to immobilized denatured S1 (negative control).

FIG. 1D shows that the result of MagMASS, used for the affinity selection and identification of cannabinoid acids (0.10 UM each in this confirmatory chromatogram) as ligands from hemp extracts.

FIG. 2A shows how CBGA is predicted to bind to an allosteric site (−6.6 kcal/mol free energy of binding).

FIG. 2B shows that, although less favorable (−6.2 kcal/mol), CBGA can also bind to the orthosteric site on the S1 C-terminal domain.

FIG. 2C shows how THCA-A is predicted to bind at the orthosteric site with free energies of binding-6.5 kcal/mol.

FIG. 2D shows how CBDA is predicted to bind at the orthosteric site with free energies of binding-6.3 kcal/mol.

FIG. 3A shows representative images of high-resolution microscopy of SARS-CoV-2 (WA1/2020) infected Vero E6 cells treated with 25 μg/mL CBDA, CBGA, or vehicle (control). FIG. 3B shows infection of ACE2 293T cells with SARS-CoV-2 spike pseudotyped lentivirus in the presence of CBDA or CBGA. FIG. 3C is a table of IC50 values for pseudovirus experiments. FIG. 3D show a live-virus infection of Vero E6 cells with SARS-CoV-2 variants (WA1/2020, B.1.1.7, and B.1.351) in the presence of CBDA. FIG. 3E show a live-virus infection of Vero E6 cells with SARS-CoV-2 variants (WA1/2020, B.1.1.7, and B.1.351) in the presence of CBGA. FIG. 3F is a table of IC50 values for live-virus experiments shown in FIGS. 3D and 3E.

FIG. 4 shows the orthosteric site residues of spike S1 receptor binding domain. The highlighted residues are mutated in the B.1.351 variant (K417N, E484K, N501Y).

FIG. 5 shows the cytotoxicity of CBDA in mammalian cell lines.

FIGS. 6A-6C show analysis of CBGA starting material: negative ion electrospray LC/MS chromatogram (6A), negative ion MS (6B), and negative ion MS/MS (6C).

FIGS. 7A-7C show analysis of CBDA starting material: negative ion electrospray LC/MS chromatogram (7A), negative ion MS (7B), and negative ion MS/MS (7C).

FIGS. 8A-8F show the high-resolution MS analysis of the methylation product of CBGA (MeCBGA): negative ion electrospray LC/MS chromatogram (8A), negative ion MS (8B), negative ion MS/MS (8C), positive ion electrospray LC/MS chromatogram (8D), positive ion MS (8E), and positive ion MS/MS (8F).

FIGS. 9A-9F show the high-resolution MS analysis of the methylation product of CBDA (MeCBDA): negative ion electrospray LC/MS chromatogram (9A), negative ion MS (9B), negative ion MS/MS (9C), positive ion electrospray LC/MS chromatogram (9D), positive ion MS (9E), and positive ion MS/MS (9F).

FIGS. 10A and 10B are LC/MS chromatograms that show that no starting material was observed in the methyl ester products synthesized from CBGA.

FIGS. 10C and 10D are LC/MS chromatograms that show that no starting material was observed in the methyl ester products synthesized from CBDA.

FIG. 11 illustrates the protocol for assessing ligand binding.

FIGS. 12A and 12B illustrate a comparison of the previous 96-well plate automated MagMASS assay (12A); and the new 5-fold faster MagMASS approach utilizing an automated magnetic particle processor (12B).

FIG. 13A illustrates that the SARS-CoV-2 is a coronavirus named for the transmembrane spike protein on the surface of the infectious virus particle.

FIG. 13B illustrates that the spike protein is a trimer consisting of a transmembrane domain, an S2 domain, and an S1 domain which binds to the human receptor ACE2 to initiate the infection of human cells.

FIG. 13C illustrates recombinant S1 protein monomer containing a histidine tag that was immobilized on magnetic beads for use as the target for MagMASS.

FIGS. 14A-14C are LC-MS chromatograms showing the results of positive ion electrospray high resolution LC-MS that was used to detect ligands that bound selectively to native SARS-CoV-2 spike S1 protein. Two hits were detected with retention times of 9.8 min and 10.9 min corresponding to elemental compositions of C₂₁H₂₂O₄ and C₂₅H₂₆O₄, respectively.

FIGS. 15A-15C show high resolution positive ion electrospray product ion tandem mass spectra of SARS-CoV-2 spike S1 protein ligands from G. inflata eluting at 9.8 min and 10.9 min during LC-MS (FIGS. 14A-14C). The peak eluting at 9.8 min was identified as licochalcone A (15A) through LC-MS/MS comparison with authentic standard (15B). The compound eluting at 10.9 min is predicted to be abyssinone III or an isomer based on dereplication (15C).

FIG. 16 illustrates the results of a MagMASS competition assay with licochalcone A standard and SBP-1 peptide incubated with SARS-CoV-2 S1 protein. The binding of licochalcone A was reduced in the presence of 2.8 μM SBP-1 indicating that both ligands compete for the orthosteric binding site on the S1 protein.

FIGS. 17A and 17B illustrate computational based modeling of the binding of licochalcone A to the SARS-CoV-2 spike S1 protein C-terminal domain (S1-CTD) orthosteric site using AutoDock Vina. The active site residues of the S1 protein are shown in FIG. 17A and for licochalcone A in FIG. 17B (predicted to bind at the orthosteric site at −6.5 kcal/mol free energy of binding).

### DETAILED DESCRIPTION

The present disclosure provides methods of treating a viral infection using cannabinoid acids. In the following description, certain specific details are set forth in order to provide a thorough understanding of various embodiments. However, one skilled in the art will understand that the invention may be practiced without these details.

Prior to setting forth this disclosure in more detail, it may be helpful to an understanding thereof to provide definitions of certain terms to be used herein. Additional definitions are set forth throughout this disclosure.

Unless the context requires otherwise, throughout the present specification and claims, the term “comprising” and variations thereof, such as “comprise” and “comprises,” are to be construed in an open, inclusive sense, synonymous with “including” and “containing,” and does not exclude additional, unrecited elements or method steps.

As used herein, the term “consisting essentially of” and variations thereof, such as “consists essentially of,” limits the scope of a claim to the specified materials or steps and those that do not materially affect the basic and novel characteristic(s) of the claimed invention. For the compositions described herein, materials such as other cannabinoids (e.g., other cannabinoid acids), hemp extracts that include cannabinoids, or therapeutic agents, are considered to materially affect the basic and novel characteristics of the compositions.

As used herein, the term “consisting of” and variations thereof, such as “consists of,” excludes any element, step, or ingredient not specified in the claim (“consisting of” is defined as closing the claim to the inclusion of materials other than those recited except for impurities ordinarily associated therewith.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless defined otherwise, all technical and scientific terms used herein have the same meaning as is commonly understood by one of skill in the art to which this invention belongs. As used in the specification and claims, the singular form “a”, “an” and “the” include plural references unless the context clearly dictates otherwise (i.e., “a” refers to one or more).

“Alkyl” refers to a saturated, straight or branched hydrocarbon chain radical consisting solely of carbon and hydrogen atoms, having from one to twelve carbon atoms (C₁-C₁₂ alkyl), preferably one to eight carbon atoms (C₁-C₈ alkyl) or one to six carbon atoms (C₁-C₆ alkyl), and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl and the like. Unless stated otherwise specifically in the specification, an alkyl group is optionally substituted.

“Alkenyl” refers to an unsaturated, straight or branched hydrocarbon chain radical consisting solely of carbon and hydrogen atoms, which contains one or more carbon-carbon double bonds), having from two to twelve carbon atoms (C₂-C₁₂ alkenyl), preferably one to two carbon atoms (C₂-C₈ alkenyl) or two to six carbon atoms (C₂-C₆ alkenyl), and which is attached to the rest of the molecule by a single bond, e.g., ethenyl, prop-1-enyl, but-1-enyl, pent-1-enyl, penta-1,4-dienyl, and the like. Unless stated otherwise specifically in the specification, an alkenyl group is optionally substituted.

“Alkynyl” refers to an unsaturated, straight or branched hydrocarbon chain radical consisting solely of carbon and hydrogen atoms, which contains one or more carbon-carbon triple bonds), having from two to twelve carbon atoms (C₂-C₁₂ alkynyl), preferably one to two carbon atoms (C₂-C₅ alkynyl) or two to six carbon atoms (C₂-C₆ alkynyl), and which is attached to the rest of the molecule by a single bond, e.g., ethynyl, propynyl, butynyl, pentynyl, hexynyl, and the like. Unless stated otherwise specifically in the specification, an alkynyl group is optionally substituted.

“Cycloalkyl” refers to a stable non-aromatic monocyclic or polycyclic carbocyclic radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7 dimethyl bicyclo[2.2.1]heptanyl, and the like. A “cycloalkenyl” is a cycloalkyl comprising one or more carbon-carbon double bonds within the ring. Unless otherwise stated specifically in the specification, a cycloalkyl (or cycloalkenyl) group is optionally substituted.

“Alkenylcycloalkenyl” refers to a radical of the formula —R_{b}R_{d} where R_{b} is cycloalkenyl as defined herein and R_{d} is an alkenyl radical as defined above. Unless stated otherwise specifically in the specification, an alkenylcycloalkenyl group is optionally substituted.

“Aromatic ring” refers to a cyclic planar portion of a molecule (i.e., a radical) with a ring of resonance bonds that exhibits increased stability relative to other connective arrangements with the same sets of atoms. Generally, aromatic rings contain a set of covalently bound co-planar atoms and comprises a number of π-electrons (for example, alternating double and single bonds) that is even but not a multiple of 4 (i.e., 4n+2 π-electrons, where n=0, 1, 2, 3, etc.). Aromatic rings include phenyl, naphthenyl, imidazolyl, pyrrolyl, pyridinyl, pyrimidinyl, pyrazinyl, pyridonyl, pyridazinyl, pyrimidonyl. Unless stated otherwise specifically in the specification, an “aromatic ring” includes all radicals that are optionally substituted.

“Heterocyclyl” or “heterocyclic ring” refers to a stable 3- to 18-membered ring radical having at least one non-aromatic ring and one to twelve ring carbon atoms (e.g., two to twelve) and from one to six ring heteroatoms (e.g., nitrogen, oxygen and sulfur). Unless stated otherwise specifically in the specification, the heterocyclyl radical is a monocyclic, bicyclic, tricyclic or tetracyclic ring system, which may include fused, spirocyclic (“spiro-heterocyclyl”) and/or bridged ring systems; and the nitrogen, carbon or sulfur atoms in the heterocyclyl radical is optionally oxidized; the nitrogen atom is optionally quaternized; and the heterocyclyl radical is partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl. 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group is optionally substituted.

The term “substituted” as used herein means any of the above groups (e.g., alkyl, alkenyl, alkynyl, cycloalkyl, cycloalkenyl, alkenylcycloalkenyl, and heterocyclyl) wherein at least one hydrogen atom (e.g., 1, 2, 3 or all hydrogen atoms) is replaced by a bond to a non-hydrogen atom such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkoxy, alkylaminyl, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an aminyl, cyano, hydroxyl, imino, nitro, oxo, thioxo, halo, alkyl, alkoxy, alkylaminyl, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl group. In addition, each of the foregoing substituents may also be optionally substituted with one or more of the above substituents.

Depending on the process conditions the end products of the present disclosure are obtained either in free (neutral) or salt form. Both the free form and the salts of these end products are within the scope of the present disclosure. If so desired, one form of a compound may be converted into another form. A free base or acid may be converted into a salt; a salt may be converted into the free compound or another salt; or a mixture of isomeric compounds may be separated into the individual isomers.

Pharmaceutically acceptable salts are preferred. However, other salts may be useful, e.g., in isolation or purification steps which may be employed during preparation and, thus, are within the scope of the present disclosure.

As used herein, “pharmaceutically acceptable salts” refer to derivatives of the disclosed compounds wherein the parent compound is modified by making acid or base salts thereof. For example, pharmaceutically acceptable salts include acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethanedisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate/hydroxymalonate, mandelate, mesylate, methylsulphate, mucate, naphthoate, napsylate, nicotinate, nitrate, octadecanoate, oleate, oxalate, palmitate, pamoate, phenylacetate, phosphate/hydrogen phosphate/dihydrogen phosphate, polygalacturonate, propionate, salicylates, stearate, succinate, sulfamate, sulfosalicylate, tartrate, tosylate, trifluoroacetate and xinafoate salt forms.

Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine. In particular embodiments, the pharmaceutically acceptable salt is a sodium salt, a potassium salt, or an ammonium salt.

The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Allen, L. V., Jr., ed., Remington: The Science and Practice of Pharmacy, 22nd Edition, Pharmaceutical Press, London, UK (2012), the disclosure of which is hereby incorporated by reference.

Conformational isomers (or conformers) are isomers that can differ by rotations about one or more bonds. Rotamers are conformers that differ by rotation about only a single bond. These terms include atropisomers.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are non-superimposable mirror images of one another.

The term “atropisomer” refers to a rotational isomer which is fixed in a particular conformation of axial or planar chirality arising from steric hinderance creating a sufficiently high barrier to the rotation necessary to achieve other conformations due to, for example, steric interference, that the particular isomer is capable of being isolated.

A “tautomer” refers to a proton shift from one atom of a molecule to another atom of the same molecule. Embodiments thus include tautomers of the disclosed compounds.

“Prodrug” is meant to indicate a compound that may be converted under physiological conditions or by solvolysis to a biologically active compound described herein (e.g., a cannabinoid acid). Thus, the term “prodrug” refers to a precursor of a biologically active compound that is pharmaceutically acceptable. In some aspects, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of a hydroxy functional group, or acetamide, formamide and benzamide derivatives of an amine functional group in the active compound and the like.

In some embodiments, prodrugs include cannabinoid acids having a phosphate, phosphoalkoxy, ester or boronic ester substituent. Without being bound by theory, it is believed that such substituents are converted to a hydroxyl group under physiological conditions. Accordingly, embodiments include any of the compounds disclosed herein, wherein a hydroxyl group has been replaced with a phosphate, phosphoalkoxy, ester or boronic ester group, for example a phosphate or phosphoalkoxy group. For example, in some embodiments a hydroxyl group on the R¹ moiety is replaced with a phosphate, phosphoalkoxy, ester or boronic ester group, for example a phosphate or alkoxy phosphate group.

The chemical naming protocol and structure diagrams used herein are a modified form of the I.U.P.A.C. nomenclature system, using the ACD/Name Version 9.07 software program and/or ChemDraw Ultra Version 11.0.1 software naming program (CambridgeSoft). For complex chemical names employed herein, a substituent group is typically named before the group to which it attaches. For example, cyclopropylethyl comprises an ethyl backbone with a cyclopropyl substituent. Except as described below, all bonds are identified in the chemical structure diagrams herein, except for all bonds on some carbon atoms, which are assumed to be bonded to sufficient hydrogen atoms to complete the valency.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

As noted above, the present disclosure is directed to cannabinoid acids and their use in various methods. As used herein, “cannabinoid” refers to compounds that bind to one or more cannabinoid receptors (e.g., cannabinoid receptor type 1 and cannabinoid receptor type 2). “Cannabinoid acid” refers to a 2-carboxylic acid of a cannabinoid.

In certain embodiments, the cannabinoid acid has the following structure (I):

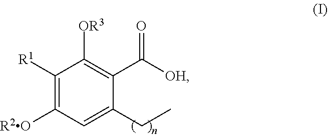

or a pharmaceutically acceptable salt, stereoisomer, or prodrug thereof, wherein:
   R¹ is alkenyl or alkenylcycloalkenyl;
   R² is H, or R¹ and R² join to form a heterocyclic ring;
   R³ is H, or R¹ and R³ join to form a heterocyclic ring;
   n is an integer ranging from 1 to 8,
   wherein each occurrence of alkenyl, alkenylcycloalkenyl, and heterocyclic ring is optionally substituted with one or more substituents unless otherwise specified.

In some embodiments, R¹ is alkenylcycloalkenyl. In some embodiments, R¹ is alkenyl. In some embodiments, R¹ and R² join to form a heterocyclic ring. In some embodiments, R³ is H. In some embodiments, R¹ and R³ join to form a heterocyclic ring. In some embodiments, R² is H. In some embodiments, the heterocyclic ring is substituted by one or more substituents selected from alkyl or alkenyl, or a combination thereof.

In some embodiments, R¹ is alkenylcycloalkenyl, R² is H, and R³ is H. In some embodiments, R¹ is alkenyl, R² is H, and R³ is H.

In some embodiments, R¹ and R² join to form a heterocyclic ring, and R³ is H. In some embodiments, R¹ and R³ join to form a heterocyclic ring, and R² is H.

In some embodiments, n is 2. In some embodiments, n is 4. In some embodiments, n is 6.

Examples of cannabinoid acids are shown in Table 1.

TABLE 1
Representative cannabinoid acids.

| Name | Structure |
|---|---|
| Cannabigerolic acid (CBGA) | 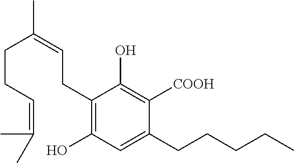 [SMILES: CCCCCc1cc(O)c(C/C=C(/C)CCC=C(C)C)c(O)c1C(=O)O] |
| Cannabidiolic acid (CBDA) | 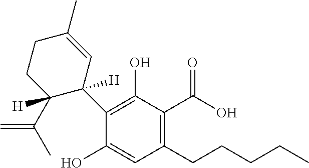 [SMILES: C=C(C)[C@@H]1CCC(C)=C[C@H]1c1c(O)cc(CCCCC)c(C(=O)O)c1O] |
| Cannabinolic acid (CBNA) | 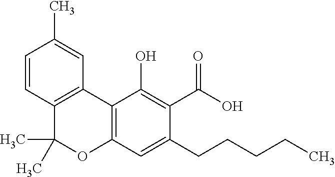 [SMILES: CCCCCc1cc2c(c(O)c1C(=O)O)-c1cc(C)ccc1C(C)(C)O2] |
| Cannabichromenic acid (CBCA) | 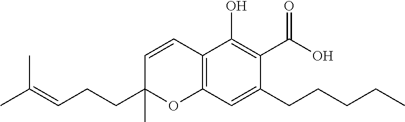 [SMILES: CCCCCc1cc2c(c(O)c1C(=O)O)C=CC(C)(CCC=C(C)C)O2] |
| Cannabichromevarinic acid (CBCVA) | 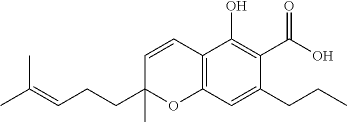 [SMILES: CCCc1cc2c(c(O)c1C(=O)O)C=CC(C)(CCC=C(C)C)O2] |
| Cannabicyclolic acid (CBNA) | 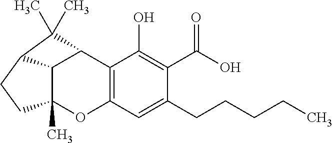 |
| Cannabidivarinic acid (CBDVA) | 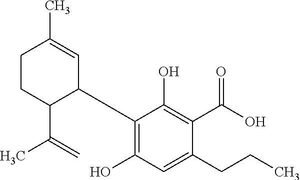 [SMILES: C=C(C)C1CCC(C)=CC1c1c(O)cc(CCC)c(C(=O)O)c1O] |
| Cannabielsoin acid A (CBEA-A) | 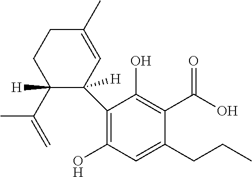 [SMILES: C=C(C)[C@@H]1CCC(C)=C[C@H]1c1c(O)cc(CCC)c(C(=O)O)c1O] |
| Cannabigerovarinic acid (CBGVA) | 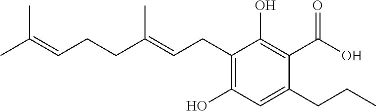 [SMILES: CCCc1cc(O)c(C/C=C(\C)CCC=C(C)C)c(O)c1C(=O)O] |
| Delta-8- tetrahydrocannabinolic acid (Δ8-THCA) | 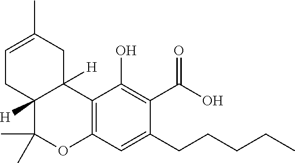 [SMILES: CCCCCc1cc2c(c(O)c1C(=O)O)C1CC(C)=CC[C@H]1C(C)(C)O2] |
| Delta-9- tetrahydrocannabinolic acid A (Δ9-THCA-A) | 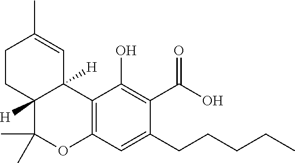 [SMILES: CCCCCc1cc2c(c(O)c1C(=O)O)[C@@H]1C=C(C)CC[C@H]1C(C)(C)O2] |
| Delta-9- tetrahydrocannabinolic acid B (Δ9-THCA-B) | 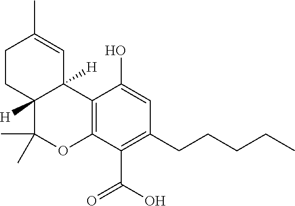 [SMILES: CCCCCc1cc(O)c2c(c1C(=O)O)OC(C)(C)[C@@H]1CCC(C)=C[C@@H]21] |
| Tetrahydrocannabinolic acid (THCA-A) | 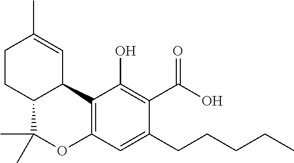 [SMILES: CCCCCc1cc2c(c(O)c1C(=O)O)[C@@H]1C=C(C)CC[C@H]1C(C)(C)O2] |
| Delta-9- tetrahydrocannabivarinic acid (Δ9-THCVA) | 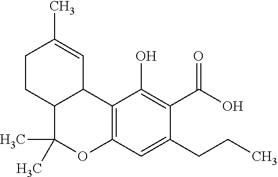 [SMILES: CCCc1cc2c(c(O)c1C(=O)O)C1C=C(C)CCC1C(C)(C)O2] |
| Tetrahydrocanabivarinic acid (THCVA) | 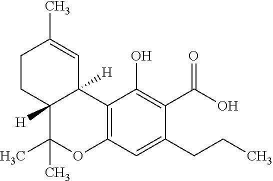 [SMILES: CCCc1cc2c(c(O)c1C(=O)O)[C@@H]1C=C(C)CC[C@H]1C(C)(C)O2] |

In some embodiments, the cannabinoid acid comprises one or more compounds of Table 1, or a pharmaceutically acceptable salt, stereoisomer, or prodrug thereof.

In embodiments, the cannabinoid acid comprises one or more of CBGA, THCA-A, CBDA, or CBNA, or a pharmaceutically acceptable salt, stereoisomer, or prodrug thereof.

In particular embodiments, the cannabinoid acid comprises CBGA, or a pharmaceutically acceptable salt, stereoisomer, or prodrug thereof. In certain embodiments, the cannabinoid acid comprises THCA-A, or a pharmaceutically acceptable salt, stereoisomer, or prodrug thereof. In specific embodiments, the cannabinoid acid comprises CBDA, or a pharmaceutically acceptable salt, stereoisomer, or prodrug thereof. In some embodiments, the cannabinoid acid comprises CBNA, or a pharmaceutically acceptable salt, stereoisomer, or prodrug thereof.

In certain embodiments, the pharmaceutically acceptable salt is a base addition salt (e.g., a sodium salt).

In another aspect, the present disclosure provides methods of treating a viral infection using cannabinoid acids. Such methods include a method for treating a coronavirus-induced disease in a subject in need thereof, the method comprising administering an effective amount of a cannabinoid acid or a pharmaceutically acceptable salt, stereoisomer, or prodrug thereof, to the subject. The methods of the disclosure further include a method for preventing infection by a coronavirus in a subject in need thereof, the method comprising administering an effective amount of a cannabinoid acid or a pharmaceutically acceptable salt, stereoisomer, or prodrug thereof, to the subject.

“Treat” or “treatment” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising a cannabinoid acid is administered in an amount sufficient to elicit a therapeutic effect or therapeutic benefit. Therapeutic effect or therapeutic benefit includes improved clinical outcome; modulation of immune response to lessen, reduce, or dampen counterproductive inflammatory cytokine activity; modulation of immune response to normalize counterproductive inflammatory cytokine activity; lessening or alleviation of symptoms associated with a disease; decreased occurrence of symptoms; improved quality of life; longer disease-free status; diminishment of extent of disease, stabilization of disease state; delay of disease progression; remission; survival; prolonged survival; or any combination thereof.

A prophylactic treatment meant to “prevent” an infection, or a disease or condition (e.g., coronavirus induced respiratory illness in a subject or patient), is a treatment administered to a subject who does not exhibit signs of a disease or exhibits only early signs, for the purpose of decreasing the risk of developing pathology or further advancement of the early disease. For example, if an individual at risk of developing a coronavirus induced disease is treated with the methods of the present disclosure and does not later develop coronavirus induced disease, then the disease has been prevented, at least over a period of time, in that individual. A prophylactic treatment can mean preventing recurrence of a disease or condition in a patient that has previously been treated for the disease or condition, e.g., by preventing relapse or recurrence of coronavirus induced disease.

A “therapeutically effective amount” or “effective amount” of a cannabinoid acid refers to an amount of a cannabinoid acid sufficient to result in a therapeutic effect, including improved clinical outcome; lessening or alleviation of symptoms associated with a disease; modulating immune response to lessen, reduce, or dampen counterproductive inflammatory cytokine activity; modulating immune response to normalize counterproductive inflammatory cytokine activity; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient or a cell expressing a single active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially or simultaneously.

In embodiments, a subject is infected with a coronavirus (e.g., a β coronavirus). In other embodiments, the subject is not infected with a coronavirus. Coronaviruses use a spike glycoprotein (comprising a S1 subunit and S2 subunit in each spike monomer) on the envelope to bind to their cellular receptors. A transmembrane protein with a molecular mass of ˜150 kDa, the spike protein forms homotrimers protruding from the SARS-CoV-2 surface. Subunits of the SARS-CoV-2 spike protein trimer consist of an S1 subunit that binds to ACE2 of host cell to initiate infection, an S2 subunit that mediates virus fusion with host cells, and a transmembrane domain.

In some embodiments, a subject is infected with a β coronavirus. In some embodiments, the β coronavirus is severe acute respiratory syndrome (SARS) coronavirus (CoV), SARS-CoV-2, or Middle East respiratory syndrome (MERS)-CoV. In particular embodiments, the β coronavirus is SARS-CoV-2. In certain embodiments, the β coronavirus is a variant of SARS-CoV-2.

In particular embodiments, the variant is a substitution in the spike protein. Examples of such variants are described in Table 2.

TABLE 2
Spike Protein Substitutions.

| Name | Spike Protein Substitutions* |
|---|---|
| B.1.525 | A67V, 69del, 70del, 144del, E484K, D614G, Q677H, F888L |
| B.1.526 | (L5F*), T95I, D253G, (S477N*), (E484K*), D614G, (A701V*) |
| B.1.526.1 | D80G, 144del, F157S, L452R, D614G, (T791I*), (T859N*), D950H |
| B.1.617 | L452R, E484Q, D614G |
| B.1.617.1 | (T95I), G142D, E154K, L452R, E484Q, D614G, P681R, Q1071H |
| B.1.617.2 | T19R, (G142D), 156del, 157del, R158G, L452R, T478K, D614G, |
| | P681R, D950N |
| B.1.617.3 | T19R, G142D, L452R, E484Q, D614G, P681R, D950N |
| P.2 | E484K, (F565L*), D614G, V1176F |
| B.1.1.7 | 69del, 70del, 144del, (E484K*), (S494P*), N501Y, A570D, D614G, |
| | P681H, T716I, S982A, D1118H (K1191N*) |
| B.1.351 | D80A, D215G, 241del, 242del, 243del, K417N, E484K, N501Y, |
| | D614G, A701V |
| B.1.427 | L452R, D614G |
| B.1.429 | S13I, W152C, L452R, D614G |
| P.1 | L18F, T20N, P26S, D138Y, R190S, K417T, E484K, N501Y, D614G, |
| | H655Y, T1027I |

*indicated relative to the sequence of the SARS-COV-2 spike protein:
MFVFLVLLPLVSSQCVNLTTRTQLPPAYTNSFTRGVYYPDKVFRSSVLHSTQDLF
LPFFSNVTWFHAIHVSGTNGTKRFDNPVLPFNDGVYFASTEKSNIIRGWIFGTTLD
SKTQSLLIVNNATNVVIKVCEFQFCNDPFLGVYYHKNNKSWMESEFRVYSSANN
CTFEYVSQPFLMDLEGKQGNFKNLREFVFKNIDGYFKIYSKHTPINLVRDLPQGFS
ALEPLVDLPIGINITRFQTLLALHRSYLTPGDSSSGWTAGAAAYYVGYLQPRTFLL
KYNENGTITDAVDCALDPLSETKCTLKSFTVEKGIYQTSNFRVQPTESIVRFPNITN
LCPFGEVENATRFASVYAWNRKRISNCVADYSVLYNSASFSTFKCYGVSPTKLN
DLCFTNVYADSFVIRGDEVRQIAPGQTGKIADYNYKLPDDFTGCVIAWNSNNLDS
KVGGNYNYLYRLFRKSNLKPFERDISTEIYQAGSTPCNGVEGENCYFPLQSYGFQ
PTNGVGYQPYRVVVLSFELLHAPATVCGPKKSTNLVKNKCVNFNFNGLTGTGVL
TESNKKFLPFQQFGRDIADTTDAVRDPQTLEILDITPCSFGGVSVITPGTNTSNQVA
VLYQDVNCTEVPVAIHADQLTPTWRVYSTGSNVFQTRAGCLIGAEHVNNSYECD
IPIGAGICASYQTQTNSPRRARSVASQSIIAYTMSLGAENSVAYSNNSIAIPTNFTIS
VTTEILPVSMTKTSVDCTMYICGDSTECSNLLLQYGSFCTQLNRALTGIAVEQDK
NTQEVFAQVKQIYKTPPIKDFGGFNFSQILPDPSKPSKRSFIEDLLENKVTLADAGF
IKQYGDCLGDIAARDLICAQKFNGLTVLPPLLTDEMIAQYTSALLAGTITSGWTFG
AGAALQIPFAMQMAYRENGIGVTQNVLYENQKLIANQFNSAIGKIQDSLSSTASA
LGKLQDVVNQNAQALNTLVKQLSSNFGAISSVLNDILSRLDKVEAEVQIDRLITG
RLQSLQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPD
VDLGDISGINASVVNIQKEIDRLNEVAKNLNESLIDLQELGKYEQYIKWPWYIWL
GFIAGLIAIVMVTIMLCCMTSCCSCLKGCCSCGSCCKFDEDDSEPVLKGVKLHYT
(SEQ ID NO: 1).

In embodiments, the variant of SARS-CoV-2 is B.1.1.7, B.1.351, P.1, B.1.427, or B.1.429. In other embodiments, the variant of SARS-CoV-2 is B.1.525, B.1.526, B.1.526.1, B.1.617, B.1.617.1, B.1.617.2, B.1.617.3, or P.2.

In some embodiments, a subject infected with a coronavirus has a coronavirus-induced disease. “Coronavirus-induced disease” refers to pathology directly resulting from SARS-CoV-1 or SARS-CoV-2 viral infection as well as immunopathology resulting from induction of a pro-inflammatory immune response. This includes secondary infections, bacterial and viral, that arise from SARS-CoV-1 and SARS-CoV-2-related immunodeficiency during the anti-viral response. In some embodiments, the coronavirus-induced disease comprises COVID-19. As used herein, “COVID-19” refers to the infectious disease caused by SARS-CoV-2 and characterized by, for example, fever, cough, respiratory symptoms, rhinorrhea, sore throat, malaise, headache, chills, repeated shaking with chills, diarrhea, new loss of smell or taste, muscle pain, or a combination thereof.

Accordingly, disclosed herein is a method for treating a β coronavirus-induced disease in a subject in need thereof, the method comprising administering an effective amount of a cannabinoid acid or a pharmaceutically acceptable salt, stereoisomer, or prodrug thereof, to the subject.

Further disclosed herein is a method for preventing infection by a β coronavirus in a subject in need thereof, the method comprising administering an effective amount of a cannabinoid acid or a pharmaceutically acceptable salt, stereoisomer, or prodrug thereof, to the subject.

In some embodiments, the subject exhibits one or more symptoms associated with mild COVID-19, moderate COVID-19, mild-to-moderate COVID-19, severe COVID-19, or exhibits no symptoms associated with COVID-19 (e.g., asymptomatic). It should be understood that in reference to the treatment of patients with different COVID-19 disease severity, “asymptomatic” infection refers to patients diagnosed with COVID-19 by a standardized RT-PCR assay that do not present with fever, cough, respiratory symptoms, rhinorrhea, sore throat, malaise, headache, or muscle pain. In some embodiments, the subject exhibits no symptom associated with COVID-19.

In some embodiments, the subject exhibits at least one symptom associated with mild COVID-19. As used herein, “mild” infection refers to patients diagnosed with COVID-19 by a standardized RT-PCR assay exhibiting fever, rhinorrhea, mild cough, sore throat, malaise, headache, muscle pain, malaise, or any combination thereof, but with no shortness of breath. Patients with “mild” infection present no signs of a more serious lower airway disease and have a respiratory rate of less than 20 breaths per minute, a heart rate of less than 90 beats per minute, and oxygen saturation (pulse oximetry) greater than 93% on room air.

In some embodiments, the subject exhibits at least one symptom associated with moderate COVID-19. As used herein, “moderate” infection refers to patients diagnosed with COVID-19 by a standardized RT-PCR assay exhibiting symptoms in the mild category and additional symptoms. These include more significant lower respiratory symptoms, including shortness of breath (at rest or with exertion) or signs of moderate pneumonia, including a respiratory rate of ≥20 but <30 breaths per minute, a heart rate of ≥90 but <125 beats per minute and oxygen saturation (pulse oximetry) greater than 93% on room air. If some embodiments, subjects with moderate infection further exhibit lung infiltrates based on X-ray or CT scan that are <50% present.

As used herein “mild-to-moderate” infection collectively refers to mild and moderate infections, as defined herein.

In some embodiments, the subject exhibits at least one symptom associated with severe COVID-19. As used herein, “severe” infection refers to patients diagnosed with COVID-19 by a standardized RT-PCR assay having significant lower respiratory symptoms, including difficulty in breathing or shortness of breath at rest or one or more of the following signs of severe pneumonia: a respiratory rate ≥30 breaths per minute, oxygen saturation (pulse oximetry) ≤93% on room air, partial pressure of oxygen/fraction of inspired oxygen (PaO2/FiO2)≤300 mmHg (1 mmHg=0.133 kPa). Additionally, clinical assessment shows evidence of rales/crackles on exam or if available, radiographic evidence of pulmonary infiltrates (chest x-ray, CT scan, etc.).

As used herein “critical” infection refers to a severe infection in which the patient has at least one of the following: (1) respiratory failure requiring at least one of the following: Endotracheal intubation and mechanical ventilation, oxygen delivered by high-flow nasal cannula, noninvasive positive pressure ventilation, or ECMO; (2) a clinical diagnosis of respiratory failure (in setting of resource limitation); (3) Septic shock (defined by SBP <90 mm Hg. or Diastolic BP<60 mm Hg); and (4) Multiple organ dysfunction/failure.

In some embodiments, the subject exhibits no symptoms associated with COVID-19 but has been exposed to another subject known or suspected of having COVID-19.

In some embodiments, the subject with a coronavirus exhibits one or more symptoms selected from dry cough, shortness of breath, and fever.

In some embodiments, treatment of a subject according to the methods described herein results in reduced duration or occurrence of hospitalization, ventilation or dialysis of the subject.

In some embodiments, treatment of a subject according to the methods described herein results in reduced lung damage to the subject.

In some embodiments, treatment of a subject according to the methods described herein results in the subject having a faster and/or more extensive recovery than a subject with similar symptoms who has not been treatment with the cannabinoid acid.

In some embodiments, treatment of a subject according to the methods described herein is not accompanied by any serious, adverse side effects.

In some embodiments, the coronavirus induced respiratory illness comprises Acute respiratory distress syndrome (ARDS). ARDS refers to a respiratory condition characterized by severe hypoxemia and may be induced by viral infection. ARDS is characterized by severe impairment in gas exchange and lung mechanics. The innate immune response plays a fundamental role in the pathophysiology of ARDS. Virally-induced inflammation promotes pulmonary epithelial and endothelial cellular damage leading to increased capillary permeability. The migration of macrophages and neutrophils and release of pro-inflammatory cytokines (cytokine storm) leads to acute respiratory distress syndrome (ARDS). Multiple immunologic processes involving neutrophils, macrophages, and dendritic cells participate in mediating tissue injury. Inflammatory injury, either locally from the lungs or systemically from extra-pulmonary sites, affect bronchial epithelium, alveolar macrophages, and vascular endothelium, causing accumulation of protein-rich edema fluid into the alveoli and, subsequently, hypoxemia due to impaired gas exchange. Alveolar macrophages play a central role in orchestrating inflammation as well as the resolution of ARDS. Once alveolar macrophages are stimulated, they recruit neutrophils and circulating macrophages to the pulmonary sites of injury. These cells produce diverse array of bioactive mediators including proteases, reactive oxygen species, eicosanoids, phospholipids, and cytokines that perpetuate inflammatory responses. Consequently, these mediators damage or induce distal cell death, specifically alveolar type 2 epithelial cells. These cells serve vital functions by synthesizing and secreting pulmonary surfactant, which is an indispensable material that lines the inner lung surface to lower alveolar surface tension. Type 2 cells also actively partake in ion transport to control lung fluid. Together, these inflammatory events lead to histological changes typical of an acute exudative phase that results in significant impairment in lung mechanics and gas exchange. During the initial inflammatory and/or resolution phases of ARDS, alveolar macrophages signal in a paracrine manner with other cells including epithelial cells, lymphocytes, and mesenchymal stem cells that can result in augmentation of the inflammatory response or accentuation of tissue injury. Patients with ARDS may exhibit buildup of fluid in the lung and have reduced oxygen levels in the blood.

In some embodiments, treatment of a subject according to the methods described herein results in reduction of the severity or duration of the severe respiratory symptoms. Severe respiratory symptoms may include, for example, shortness of breath, difficulty breathing, reduced respiratory rate, and wheezing. In some embodiments, severe respiratory symptoms comprise difficulty in breathing or shortness of breath at rest, severe pneumonia, a respiratory rate ≥30 breaths per minute, oxygen saturation (pulse oximetry) ≤93% on room air, partial pressure of oxygen/fraction of inspired oxygen (PaO2/FiO2)≤300 mmHg (1 mmHg=0.133 kPa), evidence of rales/crackles, radiographic evidence of pulmonary infiltrates (chest x-ray, CT scan, etc.), or any combination thereof.

In some embodiments, treatment of a subject according to the methods described herein results in reduced occurrence or risk of developing liver toxicity, kidney failure or a coagulation event. In some embodiments, the coagulation event comprises a blood clot, stroke, or pulmonary embolism. In some embodiments, treatment results in more normalization of kidney function. Kidney function may be measured by measuring blood levels of creatine, BUN, sodium, or any combination thereof in the subject blood. In some embodiments, treating results in more normalization of liver function. Liver function may be measured by measuring blood levels of bilirubin, alanine transaminase (ALT), aspartate aminotransferase (AST), or any combination thereof.

In some embodiments, treatment of a subject according to the methods described herein results in reduction of SARS-CoV-2 viral load in the subject. In some embodiments, the reduction in plasma SARS-CoV-2 viral load occurs by day 7 of treatment. In some embodiments, the plasma SARS-CoV-2 viral load is measured by droplet digital PCR. In some embodiments, the plasma SARS-CoV-2 viral load is measured by detecting the nucleocapsid (N) gene.

In certain embodiments, the disclosure provides a method for preventing infection by a β coronavirus in a subject or treating a β coronavirus-induced disease in a subject in need thereof, the method comprising administering an effective amount of a cannabinoid acid (i.e., one or more cannabinoid acids) or a pharmaceutically acceptable salt, stereoisomer, or prodrug thereof, to the subject, wherein the cannabinoid acid has formula (II):

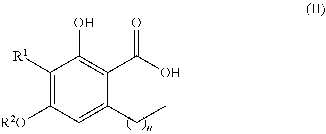

or a pharmaceutically acceptable salt, stereoisomer, or prodrug thereof, wherein
R¹ is alkenyl or alkenylcycloalkenyl;
R² is H or R¹ and R² join to form a tetrahydro- or dihydropyan ring;
n is an integer from 1 to 8,
wherein each occurrence of alkenyl and alkenylcycloalkenyl is optionally substituted with one or more substituents.

In certain of these embodiments, the method comprises administering to the subject an effective amount of a composition comprising, consisting essentially of, or consisting of a cannabinoid acid (i.e., one or more cannabinoid acids), or a pharmaceutically acceptable salt, stereoisomer, or prodrug thereof, wherein the cannabinoid acid has formula (II), and optionally further includes a pharmaceutically acceptable carrier. It will be appreciated that in certain of these embodiments, the cannabinoid acid composition is in the form of a pharmaceutical composition that includes a pharmaceutically acceptable carrier.

In certain embodiments, the above method consists essentially of the specified steps. In other embodiments, the above method consists of the specified steps.

In certain of these embodiments, R¹ is alkenylcycloalkenyl. Representative compounds where R¹ is alkenylcycloalkenyl include CBDA, CBDVA, and CBEA-A.

In other of these embodiments, R¹ is alkenyl. Representative compounds where R¹ is alkenyl include CBGA and CBGVA.

In certain embodiments, R¹ and R² join to form a tetrahydro- or dihydropyan ring (i.e., R¹ and R² taken together with the atoms to which they are attached form a tetrahydro- or dihydropyan ring). Representative compounds where R¹ and R² join to form a tetrahydro- or dihydropyan ring include CBCA, CBCVA, THCAs, and THCVAs.

In certain embodiments for compounds of formula (II), R² is H.

In certain embodiments for compounds of formula (II), the tetrahydro- or dihydropyan ring is substituted by one or more substituents selected from alkyl or alkenyl, or a combination thereof.

In certain embodiments for compounds of formula (II), n is 2. In other embodiments for compounds of formula (II), n is 4. In further embodiments for compounds of formula (II), n is 6.

In certain embodiments, for compounds of formula (II), the cannabinoid acid is a compound of Table 1, or a pharmaceutically acceptable salt, stereoisomer, or prodrug thereof.

In certain embodiments for compounds of formula (II), the cannabinoid acid is one or more of cannabigerolic acid (CBGA), tetrahydrocannabinolic acid (THCA-A), cannabidiolic acid (CBDA), or cannabinolic acid (CBNA), or pharmaceutically acceptable salts, stereoisomers, or prodrugs thereof.

In certain of these embodiments, the cannabinoid acid is CBDA or a pharmaceutically acceptable salt, stereoisomer, or prodrug thereof.

In other of these embodiments, the cannabinoid acid is CBGA or a pharmaceutically acceptable salt, stereoisomer, or prodrug thereof.

In further of these embodiments, the cannabinoid acid is THCA-A or a pharmaceutically acceptable salt, stereoisomer, or prodrug thereof.

In yet other of these embodiments, the cannabinoid acid is CBNA or a pharmaceutically acceptable salt, stereoisomer, or prodrug thereof.

It will be appreciated that for the methods described herein that specify administering to a subject an effective amount of a cannabinoid acid (i.e., one or more cannabinoid acids) or a pharmaceutically acceptable salt, stereoisomer, or prodrug thereof, the cannabinoid acid is not a hemp extract or other processed hemp composition (e.g., a mixture of one or more cannabinoid acids and/or other cannabinoids). The methods described herein do not use or contemplate the use of a hemp extract or the like that include mixtures of cannabinoid acids and/or other cannabinoids.

### Specific Advantageous Cannabinoid Acid Combinations: CBGA and CBDA and CBGA and THCA-A

Hemp (Cannabis sativa L.) compounds cannabigerolic acid (CBGA), cannabidiolic acid (CBDA), and Δ⁹-tetrahydrocannabinolic acid-A (THCA-A) are orthosteric ligands of the SARS-CoV-2 spike protein (with micromolar Kd) and can prevent infection of human cells. CBGA is also an allosteric ligand of the spike protein. Specifically, THCA-A and CBDA bind orthosterically (that is to the region of the virus spike protein that binds to the ACE2 receptor of human cells) whereas CBGA can bind allosterically (a site remote from the orthosteric site). Therefore, CBGA can bind simultaneously with either THCA-A or CBDA to inhibit virus cell entry (van Breemen R B, Muchiri R N, Bates T A, Weinstein J B, Leier H C, Farley S, Tafesse F G. Cannabinoids block cellular entry of SARS-CoV-2 and the emerging variants. J. Nat. Prod. 2022; 85: 176-184. doi: 10.1021/acs.jnatprod.1c00946).

Therefore, the present disclosure provides cannabinoid acid combinations to advantageously target SARS-CoV-2 spike protein via orthosteric binding and allosteric binding. In certain embodiments of the above method, the cannabinoid acid is a combination of CBDA, or pharmaceutically acceptable salt, stereoisomer, or prodrug thereof, and CBGA, or pharmaceutically acceptable salt, stereoisomer, or prodrug thereof. In other embodiments of the above method, the cannabinoid acid is a combination of THCA-A, or pharmaceutically acceptable salt, stereoisomer, or prodrug thereof, and CBGA, or pharmaceutically acceptable salt, stereoisomer, or prodrug thereof.

### Specific Advantageous Combinations of a Cannabinoid Acid and Transporter Inhibitors: CBDA and CBD or CBG.

Cannabidiol (CBD) and cannabigerol (CBG) are inhibitors of the transporter ABCG2 (also called BCRP) (Lyndsey L Anderson L L, Etchart M G, Bahceci D, Golembiewski T A, Arnold J C. Cannabis constituents interact at the drug efflux pump BCRP to markedly increase plasma cannabidiolic acid concentrations. Sci. Rep. 2021 Jul. 22; 11:14948. doi: 10.1038/s41598-021-94212-6), which is an efflux transporter that restricts distribution of substrates into the brain and absorption from the gastrointestinal tract (Gomez-Zepeda D. Taghi M, Scherrmann J-M, Decleves X, Menet M-C. ABC transporters at the blood-brain interfaces, their study models, and drug delivery implications in gliomas. Pharmaceutics, 2020; 12: 20. doi: 10.3390/pharmaceutics 12010020). The ABCG2 transporter can block absorption of drugs at the apical membrane of the intestine and at the blood-brain barrier (Vlaming M L, Lagas J S. Schinkel A H. Physiological and pharmacological roles of ABCG2 (BCRP): recent findings in Abcg2 knockout mice. Adv. Drug Delivery Rev. 2009; 61: 14-25). The ABCG2 transporter also enhances excretion of xenobiotics at the apical membranes of the liver and kidney, thereby enhancing their excretion into bile or urine, respectively.

CBDA is a substrate for the efflux transporter ABCG2, which will reduce its absorption from the intestinal tract, prevent it from entering the brain, and decrease its plasma half-life by enhancing it biliary and urinary excretion. CBD and CBG enhance the bioavailability of CBDA by blocking ABCG2. CBD and CBG also prolong the half-life of CBDA by blocking ABCG2, which would otherwise pump CBDA out of the body into the bile or, to a lesser extent, urine.

Therefore, the present disclosure provides combinations of a cannabinoid acid and transport inhibitors to improve the action of the cannabinoid acid. In certain embodiments of the above method, the cannabinoid acid is CBDA, or pharmaceutically acceptable salt, stereoisomer, or prodrug thereof, and wherein administering an effective amount of a cannabinoid acid or a pharmaceutically acceptable salt, stereoisomer, or prodrug thereof, further comprises administering to the subject an effective amount a compound that inhibits (efflux) transport of CBDA out of cells. In certain of these embodiments, the compound that inhibits transport of CBDA out of cells is CBG and/or CBD.

A cannabinoid acid may be formulated in any suitable manner. Accordingly, provided herein are pharmaceutical compositions comprising a cannabinoid acid or a pharmaceutically acceptable salt, stereoisomer, or prodrug thereof.

A cannabinoid acid may be formulated in any suitable manner. Accordingly, provided herein are pharmaceutical compositions comprising a cannabinoid acid or a pharmaceutically acceptable salt, stereoisomer, or prodrug thereof.

A “pharmaceutical composition” refers to a formulation of a cannabinoid acid or a pharmaceutically acceptable salt, stereoisomer, or prodrug thereof and a medium generally accepted in the art for the delivery of the biologically active compound to mammals, e.g., humans. Such a medium includes all pharmaceutically acceptable carriers, diluents, or excipients therefor.

“Pharmaceutically acceptable carrier, diluent or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The compositions may comprise different types of pharmaceutically acceptable carriers, diluents, or excipients depending on the desired mode of administration (e.g., solid, liquid or aerosol form). Cannabinoid acids can be administered intranasally, mucosally, orally, topically, locally, by inhalation (e.g., aerosol inhalation), or by other methods or any combination of the forgoing as would be understood by one of ordinary skill in the art (see, e.g., Remington's Pharmaceutical Sciences, 18th Ed. Mack Printing Company, 1990).

In another aspect, the present disclosure provides pharmaceutical compositions that include a cannabinoid acid (i.e., one or more cannabinoid acids). In certain embodiments, the pharmaceutical composition includes a cannabinoid acid of formula (I), or pharmaceutically acceptable salt, stereoisomer, or prodrug thereof. In other embodiments, the pharmaceutical composition includes a cannabinoid acid of formula (II), or pharmaceutically acceptable salt, stereoisomer, or prodrug thereof.

In certain embodiments, the pharmaceutical composition comprises a cannabinoid acid of formula (I) or formula (II), or pharmaceutically acceptable salt, stereoisomer, or prodrug thereof. In other embodiments, the pharmaceutical composition consists essentially of a cannabinoid acid of formula (I) or formula (II), or pharmaceutically acceptable salt, stereoisomer, or prodrug thereof. In further embodiments, the pharmaceutical composition consists of a cannabinoid acid of formula (I) or formula (II), or pharmaceutically acceptable salt, stereoisomer, or prodrug thereof.

It will be appreciated that the compositions described herein that include a cannabinoid acid (i.e., one or more cannabinoid acids), or a pharmaceutically acceptable salt, stereoisomer, or prodrug thereof, are not and do not include a hemp extract or other processed hemp composition (e.g., a mixture of one or more cannabinoid acids and/or other cannabinoids).

In certain embodiments, the pharmaceutical composition comprises CBDA, or pharmaceutically acceptable salt, stereoisomer, or prodrug thereof, and CBGA, or a pharmaceutically acceptable salt, stereoisomer, or prodrug thereof, and a pharmaceutically acceptable carrier. In other of these embodiments, the pharmaceutical composition consists essentially of CBDA, or pharmaceutically acceptable salt, stereoisomer, or prodrug thereof, and CBGA, or a pharmaceutically acceptable salt, stereoisomer, or prodrug thereof, and a pharmaceutically acceptable carrier. In further of these embodiments, the pharmaceutical composition consists of CBDA, or pharmaceutically acceptable salt, stereoisomer, or prodrug thereof, and CBGA, or a pharmaceutically acceptable salt, stereoisomer, or prodrug thereof, and a pharmaceutically acceptable carrier.

In other embodiments, the pharmaceutical composition comprises CBDA, or a pharmaceutically acceptable salt, stereoisomer, or prodrug thereof, and a compound that inhibits transport of CBDA out of cells, and a pharmaceutically acceptable carrier. In certain of these embodiments, the compound that inhibits transport of CBDA out of cells is CBG and/or CBD.

Therefore, in certain embodiments, the pharmaceutical composition comprises CBDA, or pharmaceutically acceptable salt, stereoisomer, or prodrug thereof, and CBG and/or CBD, or a pharmaceutically acceptable salt, stereoisomer, or prodrug thereof, and a pharmaceutically acceptable carrier. In other embodiments, the pharmaceutical composition consists essentially of CBDA, or pharmaceutically acceptable salt, stereoisomer, or prodrug thereof, and CBG and/or CBD, or a pharmaceutically acceptable salt, stereoisomer, or prodrug thereof, and a pharmaceutically acceptable carrier. In further embodiments, the pharmaceutical composition consists of CBDA, or pharmaceutically acceptable salt, stereoisomer, or prodrug thereof, and CBG and/or CBD, or a pharmaceutically acceptable salt, stereoisomer, or prodrug thereof, and a pharmaceutically acceptable carrier.

A “pharmaceutical composition” refers to a formulation of a cannabinoid acid or a pharmaceutically acceptable salt, stereoisomer, or prodrug thereof and a medium generally accepted in the art for the delivery of the biologically active compound to mammals, e.g., humans. Such a medium includes all pharmaceutically acceptable carriers, diluents, or excipients therefor.

“Pharmaceutically acceptable carrier, diluent or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The compositions may comprise different types of pharmaceutically acceptable carriers, diluents, or excipients depending on the desired mode of administration (e.g., solid, liquid or aerosol form). Cannabinoid acids can be administered intranasally, mucosally, orally, topically, locally, by inhalation (e.g., aerosol inhalation), or by other methods or any combination of the forgoing as would be understood by one of ordinary skill in the art (see, e.g., Remington's Pharmaceutical Sciences, 18th Ed. Mack Printing Company, 1990).

Cannabinoid acids may be administered using any suitable route of administration (e.g., oral, intravenous, aerosol, parenteral, ophthalmic, pulmonary, transmucosal, transdermal, otic, nasal, and topical administration). In embodiments, the cannabinoid acid or the pharmaceutically acceptable salt, stereoisomer, or prodrug thereof, is administered orally. In some embodiments, the cannabinoid acid or the pharmaceutically acceptable salt, stereoisomer, or prodrug thereof, is administered topically. In further embodiments, the cannabinoid acid or the pharmaceutically acceptable salt, stereoisomer, or prodrug thereof, is inhaled.

In another embodiment, the cannabinoid acid or the pharmaceutically acceptable salt, stereoisomer, or prodrug thereof, described herein are formulated for oral administration. Compounds described herein are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In other embodiments, therapeutically effective amounts of at least one of the compounds described herein are formulated for buccal or sublingual administration. Formulations suitable for buccal or sublingual administration include, by way of example only, tablets, lozenges, or gels. In still other embodiments, the compounds described herein are formulated for parenteral injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of the active compounds (e.g., the cannabinoid acid or the pharmaceutically acceptable salt, stereoisomer, or prodrug thereof) are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

In still other embodiments, the cannabinoid acid or the pharmaceutically acceptable salt, stereoisomer, or prodrug thereof, are administered topically. The compounds described herein are formulated into a variety of topically administrable compositions, such as solutions, suspensions, lotions, gels, pastes, medicated sticks, balms, creams, or ointments. Such pharmaceutical compositions optionally contain solubilizers, stabilizers, tonicity enhancing agents, buffers, and preservatives.

In yet other embodiments, the cannabinoid acid or the pharmaceutically acceptable salt, stereoisomer, or prodrug thereof, are formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of the cannabinoid acid or the pharmaceutically acceptable salt, stereoisomer, or prodrug thereof, is accomplished by means of iontophoretic patches and the like. In certain embodiments, transdermal patches provide controlled delivery of the cannabinoid acid or the pharmaceutically acceptable salt, stereoisomer, or prodrug thereof. In specific embodiments, the rate of absorption is slowed by using rate-controlling membranes or by trapping the compound within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In other embodiments, the cannabinoid acid or the pharmaceutically acceptable salt, stereoisomer, or prodrug thereof, are formulated for administration by inhalation. Various forms suitable for administration by inhalation include, but are not limited to, aerosols, mists or powders. Pharmaceutical compositions of any of the cannabinoid acid or the pharmaceutically acceptable salt, stereoisomer, or prodrug thereof, are conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatin for use in an inhaler or insufflator is formulated containing a powder mix of the compound and a suitable powder base such as lactose or starch.

The cannabinoid acid or the pharmaceutically acceptable salt, stereoisomer, or prodrug thereof, is effective over a wide dosage range. For example, in the treatment of adult humans, dosages from 0.01 to 1000 mg, from 0.5 to 100 mg, from 1 to 50 mg per day, and from 5 to 40 mg per day are examples of dosages that are used in some embodiments. In embodiments, the cannabinoid acid or the pharmaceutically acceptable salt, stereoisomer, or prodrug thereof, is administered in a dose of at least 0.5 mg/kg. In some embodiments, the cannabinoid acid or the pharmaceutically acceptable salt, stereoisomer, or prodrug thereof, is administered in a dose of at least 1 mg/kg. An exemplary dosage is 10 to 30 mg per day. Further exemplary dosages include 0.5 to 20 mg/kg and 1 to 15 mg/kg.

In some embodiments, the cannabinoid acid or the pharmaceutically acceptable salt, stereoisomer, or prodrug thereof, is administered in a mixture of a plurality of cannabinoid acids, wherein each of cannabinoid acid of the plurality of cannabinoid acids is administered in a dose of at least 0.5 mg/kg. In particular embodiments, the dose is at least 1 mg/kg. In certain embodiments, the dose ranges from 0.5 to 20 mg/kg. In further embodiments, the dose ranges from 1 to 15 mg/kg.

The exact dosage will depend upon the route of administration, the form in which the compound is administered, the subject to be treated, the body weight of the subject to be treated, and the preference and experience of the attending physician.

In some embodiments, the cannabinoid acid or the pharmaceutically acceptable salt, stereoisomer, or prodrug thereof, is administered in a single dose. Typically, such administration will be by injection, e.g., intravenous injection, in order to introduce the agent quickly. However, other routes are used as appropriate. A single dose of a cannabinoid acid or the pharmaceutically acceptable salt, stereoisomer, or prodrug thereof, may also be used for treatment of an acute condition.

In some embodiments, the cannabinoid acid or the pharmaceutically acceptable salt, stereoisomer, or prodrug thereof, is administered in multiple doses. In some embodiments, dosing is about once, twice, three times, four times, five times, six times, or more than six times per day. In other embodiments, dosing is about once a month, once every two weeks, once a week, or once every other day. In another embodiment a cannabinoid acid or the pharmaceutically acceptable salt, stereoisomer, or prodrug thereof, and another agent are administered together about once per day to about 6 times per day. In another embodiment the administration of a cannabinoid acid or the pharmaceutically acceptable salt, stereoisomer, or prodrug thereof, and an agent continues for less than about 7 days. In yet another embodiment the administration continues for more than about 6, 10, 14, 28 days, two months, six months, or one year. In some cases, continuous dosing is achieved and maintained as long as necessary.

Administration of the cannabinoid acid or the pharmaceutically acceptable salt, stereoisomer, or prodrug thereof, may continue as long as necessary. In some embodiments, a cannabinoid acid or the pharmaceutically acceptable salt, stereoisomer, or prodrug thereof, is administered for more than 1, 2, 3, 4, 5, 6, 7, 14, or 28 days. In some embodiments, a cannabinoid acid or the pharmaceutically acceptable salt, stereoisomer, or prodrug thereof, is administered for less than 28, 14, 7, 6, 5, 4, 3, 2, or 1 day. In some embodiments, a cannabinoid acid or the pharmaceutically acceptable salt, stereoisomer, or prodrug thereof, is administered chronically on an ongoing basis, e.g., for the treatment of chronic effects.

In some embodiments, the cannabinoid acids or the pharmaceutically acceptable salt, stereoisomer, or prodrug thereof, are administered in dosages. It is known in the art that due to intersubject variability in compound pharmacokinetics, individualization of dosing regimen is necessary for optimal therapy. Dosing for a cannabinoid acid or the pharmaceutically acceptable salt, stereoisomer, or prodrug thereof, may be found by routine experimentation in light of the instant disclosure.

In certain embodiments, the methods of the present disclosure include administration of a cannabinoid acid provided herein to a subject in combination with one or more additional therapeutic agents. In some embodiments, the one or more additional therapeutic agents comprises an antiviral agent, an immune checkpoint molecule inhibitor, or any combination thereof. In some embodiments, the one or more additional therapeutic agents is administered simultaneously, separately, or sequentially with the cannabinoid acid.

As used herein, the term “immune checkpoint molecule” refers to one or more proteins, molecules, compounds, or complexes providing inhibitory signals to assist in controlling or suppressing an immune response. For example, immune checkpoint molecules include those molecules that partially or totally block immune stimulation; decrease, prevent or delay immune activation; or increase, activate, or up regulate immune suppression. Exemplary immune checkpoint molecules are described in further detail herein and include PD-1, PD-L1, PD-L2, CD80, CD86, B7-H3, B7-H4, HVEM, adenosine, GAL9, VISTA, CEACAM-1, PVRL2, CTLA-4, BTLA, KIR, LAG3, TIM3, A2aR, CD244/2B4, CD160, TIGIT, LAIR-1, PVRIG/CD112R, and certain metabolic enzymes, such as arginase, indoleamine 2,3-dioxygenase (IDO).

As used herein, the term “antiviral agents” refers to a class of drugs for treating viral infections. An antiviral agent may be, for example, a small molecule, peptide, protein, antibody, nucleic acid, or aptamer that targets one or more components in the viral life cycle: attachment to host cell; release of viral genes and possibly enzymes into the host cell; replication of viral components using host cell machinery; assembly of viral components into viral particles; and release of viral particles to infect new host cells. Targets of antiviral agents include critical viral proteins such as neuraminidase, M2 ion channel protein, hemagglutinin, viral RNA polymerase, NTPase/helicase, spike (S) glycoprotein (S1 domain), and 3C-like cysteine protease. Examples of anti-viral agents include seltamivir, zanamivir, laninamivir, laninamivir, peramivir, and remdesivir. Other compounds with antiviral properties include chloroquine and hydroxychloroquine.

An immune checkpoint molecule inhibitor may be a compound, an antibody, an antibody fragment or fusion polypeptide (e.g., Fc fusion, such as CTLA4-Fc or LAG3-Fc), an antisense molecule, a ribozyme or RNAi molecule, or a low molecular weight organic molecule. In some embodiments, a cannabinoid acid is administered in combination with a PD-1 inhibitor, for example a PD-1-specific antibody or binding fragment thereof, such as pidilizumab, nivolumab (Keytruda, formerly MDX-1106), pembrolizumab (Opdivo, formerly MK-3475), MEDI0680 (formerly AMP-514), AMP-224, BMS-936558 or any combination thereof. In some embodiments, a cannabinoid acid is administered in combination with a PD-L1 specific antibody or binding fragment thereof, such as BMS-936559, durvalumab (MEDI4736), atezolizumab (RG7446), avelumab (MSB0010718C), MPDL3280A, or any combination thereof. In some embodiments, a cannabinoid acid is administered in combination with an inhibitor of CTLA4, such as ipilimumab, tremelimumab, CTLA4-Ig fusion proteins (e.g., abatacept, belatacept), or any combination thereof.

In another aspect, the present disclosure provides methods of screening compounds. Such methods include a method for identifying a ligand that binds to a protein (e.g., a SARS-CoV-2 spike protein).

In such methods, a protein (e.g., a SARS-CoV-2 spike protein) is immobilized on a magnetic bead. Any suitable protein, including natural and variant spike proteins may be used, and any suitable methods of immobilization may be used such that after immobilization, the protein maintains activity. In embodiments, the protein comprises a recombinant SARS-CoV-2 spike protein S1 subunit (˜72 kDa) containing an N-terminal His-tag. Further, any suitable magnetic beads may be used. In specific embodiments, the beads are Ni²⁺-nitrilotriacetic acid derivatized magnetic microbeads.

The immobilized proteins and beads are mixed with potential ligands of interest in a well or other suitable container. After sufficient time for the ligand(s) to bind to the immobilized protein, a magnet is introduced into the well. In embodiments, a ThermoFisher KingFisher Flex Magnetic Particle Processor is used.

Any unbound ligands are then removed by rinsing. The bound ligands are then released, e.g., using a denaturing solvent. The released ligands are then assessed using, for example, liquid chromatography-mass spectrometry (LC-MS).

Accordingly, described herein is a method for identifying a ligand that binds to SARS-CoV-2 spike protein, comprising: mixing, in a well, the ligand and the SARS-CoV-2 spike protein such that the ligand binds to the SARS-CoV-2 spike protein, wherein the SARS-CoV-2 spike protein has been immobilized on a magnetic bead; capturing the ligand bound to the SARS-CoV-2 spike protein by introducing a magnet into the well; releasing the ligand; and analyzing the ligand using LC-MS.

### EXAMPLES

### Example 1

Cannabinoid Acids that Block Cellular Entry of SARS-Cov-2 and the Emerging Variants

A member of the Coronaviridae family, SARS-CoV-2 is an enveloped, non-segmented, positive sense RNA virus that is characterized by crown-like spikes on the outer surface. SARS-CoV-2 contains RNA strands 29.9 kb long that encode the 4 main structural proteins spike, envelope, membrane, and nucleocapsid, 16 nonstructural proteins, and several accessory proteins.

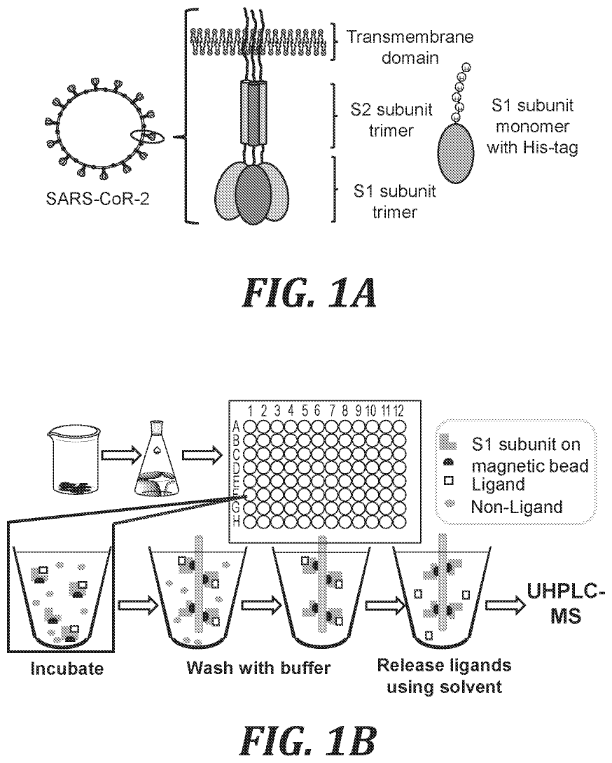

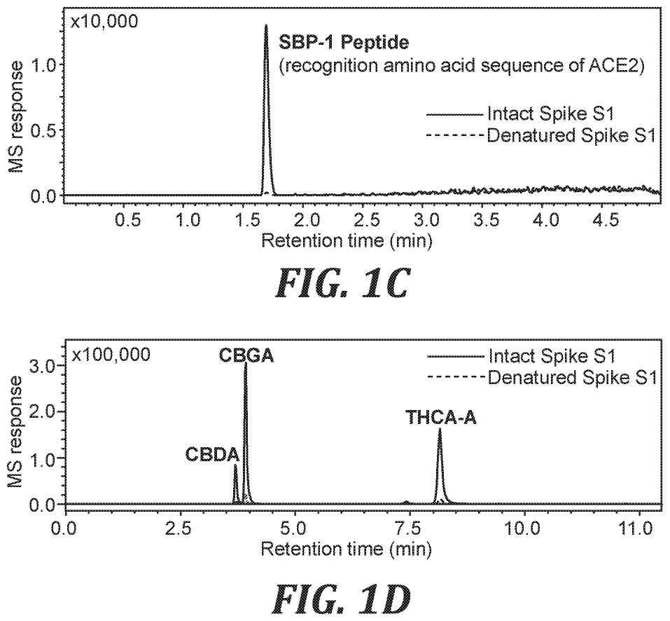

A transmembrane protein with a molecular mass of ˜150 kDa, the spike protein forms homotrimers protruding from the SARS-CoV-2 surface. Subunits of the SARS-CoV-2 spike protein trimer consist of an S1 subunit that binds to ACE2 of host cell to initiate infection, an S2 subunit that mediates virus fusion with host cells, and a transmembrane domain (FIG. 1A). The infection of host cells by SARS-CoV-2 begins with the attachment of the receptor-binding domain (RBD) of the S1 protein, which has been identified as residues 331 to 524, to the host cell receptor ACE2. An enzyme on the outer cell membrane of host cells, ACE2 is expressed abundantly on human endothelial cells in the lungs, arteries, heart, kidney, and intestines. Following membrane fusion, TMPRSS2 protease on the host cell membrane activates the spike protein by cleaving it at S1/S2 and S2 sites, leading to conformational changes that allow the virus to enter the host cell. The S1 subunit is primarily responsible for the determination of the host virus range and cellular tropism.

Ligands with high affinity to the receptor binding domain on the S1 protein have the potential to function as entry inhibitors and prevent infection of human cells by SARS-CoV-2.

Although bioassay-guided fractionation is widely used for natural products drug discovery, affinity selection-mass spectrometry (AS-MS) provides a more efficient alternative.2 AS-MS involves incubating a therapeutically important receptor like the SARS-CoV-2 spike protein with a mixture of possible ligands such as a botanical extract. The ligand-receptor complexes are separated from non-binding molecules using one of several methods such as ultrafiltration, size exclusion or magnetic microbeads, and then ultrahigh-pressure liquid chromatography-mass spectrometry (UHPLC-MS) is used to characterize the affinity-extracted ligands. The AS-MS approach of magnetic microbead affinity selection screening (MagMASS), as described in Example 3, was used.

Hemp (Cannabis sativa L.) has over 170 secondary metabolites including flavonoids, diterpenes, triterpenes, lignans, and cannabinoids. There are at least 70 cannabinoids including cannabidiols, 49-tetrahydrocannabinols, 48-tetrahydrocannabinols, cannabigerols, cannabinols, cannabichromenes, and cannabitriols.

Using MagMASS to screen hemp extracts for ligands to the SARS-CoV-2 spike protein, several cannabinoid ligands were identified and ranked by affinity to the spike protein. Two cannabinoids with the highest affinities for the spike protein, cannabidiolic acid (CBDA) and cannabigerolic acid (CBGA), were confirmed to block infection of human epithelial cells by a pseudovirus expressing the spike protein. Both CBDA and CBGA block infection of the original live SARS-CoV-2 virus and the variants of concern, including the B.1.1.7 and B.1.351.

To discover natural ligands to the SARS-CoV-2 spike protein, a MagMASS assay was developed using the spike protein S1 subunit immobilized on magnetic microbeads (FIG. 1). To confirm that the immobilized S1 subunit retained selectivity for the human cell surface protein ACE2, magnetic microbeads containing the S1 subunit were incubated with SBP-1, which is a peptide containing the amino acid sequence of human ACE2 to which the SARS-CoV-2 virus spike protein recognition site binds (amino acid residues 331 to 524). After processing using MagMASS (FIG. 1B), SBP-1 showed selective binding to the immobilized spike protein S1 subunit (positive control) whereas SBP-1 did not bind to the denatured S1 subunit that had been immobilized in an identical manner (FIG. 1C).

During screening of botanical extracts using MagMASS, extracts of hemp (Cannabis sativa L.) produced several hits (FIG. 1D). FIG. 1 describes the affinity selection-mass spectrometric (AS-MS) discovery of natural ligands to the SARS-CoV-2 spike protein. The spike protein of SARS-CoV-2 consists of trimers of a protein containing an S1 subunit, an S2 subunit, and a transmembrane domain (FIG. 1A). The S1 subunit binds to human ACE2 to initiate cell entry. Recombinant S1 containing a His-tag was immobilized on magnetic microbeads for affinity selection of ligands. AS-MS was used to isolate and identify natural ligands to the spike protein S1 subunit (FIG. 1B). A magnetic probe retained the microbeads containing the S1 subunit and bound ligands while unbound compounds were washed away. Ligands were released using organic solvent and then analyzed using UHPLC-MS. During AS-MS, the SBP-1 peptide bound to immobilized S1 (equivalent to 0.17 μM) (positive control) but not to immobilized denatured S1 (negative control) (FIG. 1C). MagMASS was used for the affinity selection and identification of cannabinoid acids (0.10 μM each in this confirmatory chromatogram) as ligands from hemp extracts (FIG. 1D). Negative controls using denatured S1 showed no significant binding of cannabinoid acids.

Based on dereplication of the hits with and follow-up assays using cannabinoid standards, the spike protein ligands with the highest binding affinities (as indicated by the fold peak area enrichment) were identified as CBGA, THCA-A, CBDA, and CBNA. The d9-tetrahydrocannabinol, d8-tetrahydrocannabinol, cannabichromene, cannabinoids cannabigerol, cannabinol, and cannabidiol showed only weak or no binding based on competitive binding MagMASS assays using equimolar mixtures (Table 3). Higher fold peak area enrichment indicates a higher affinity (lower Kd). In this experiment, 2-fold and 3-fold enhancement was judged to be essentially background noise (i.e., weak or no binding) when compared with 10-fold and 20-fold enhancement for the highest affinity ligands.

Dissociation constants and ligand docking. The Kd values for the binding of CBGA and CBDA to the SARS-CoV-2 spike protein S1 subunit were determined using equilibrium dialysis. The optimum time for full equilibration of CBGA was 5 h while that of CBDA was 4 h. The Kd values for CBGA and CBDA were 19.8±2.7 μM and 5.6±2.2 μM, respectively. Because THCA-A is a controlled substance, insufficient quantities were available for determination of binding affinity or anti-viral activity.

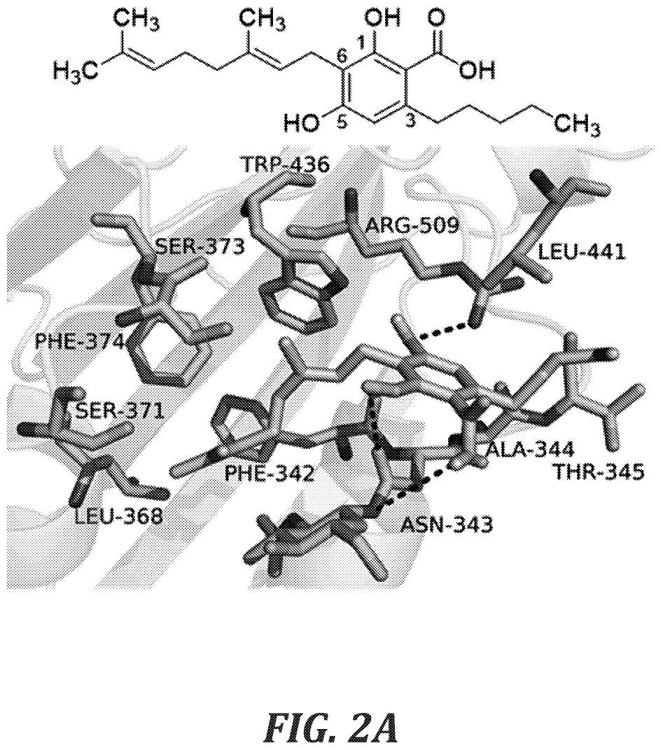

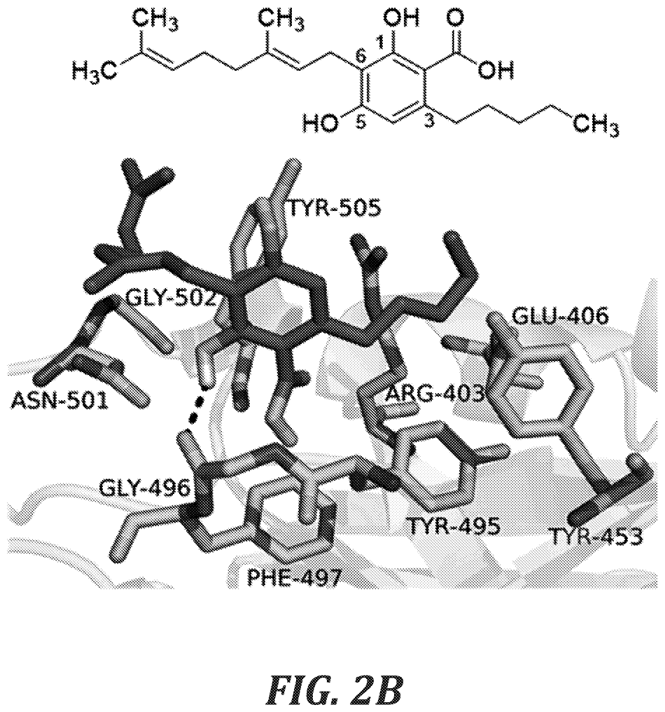

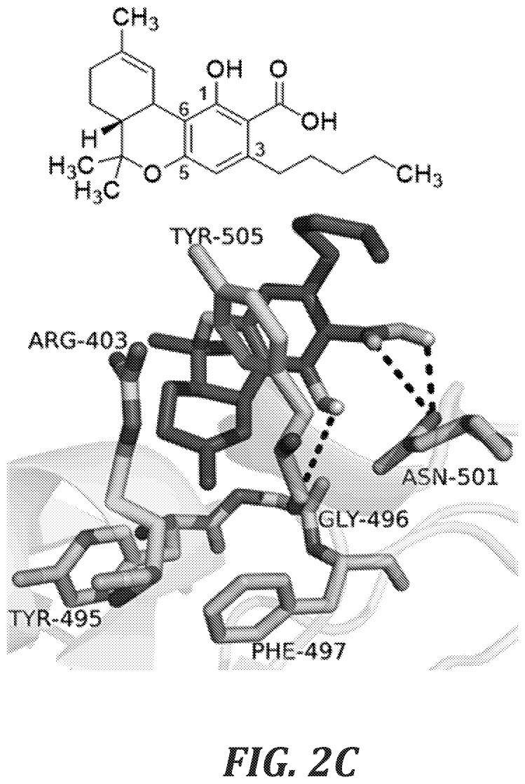

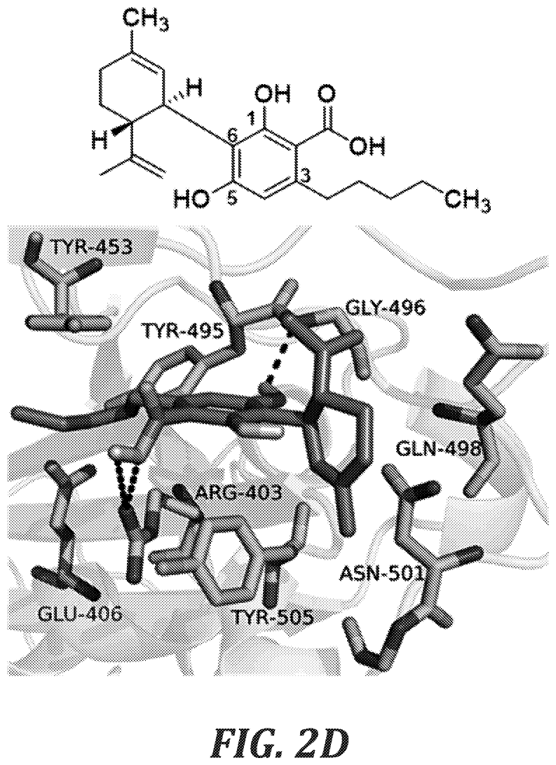

The binding interactions of CBDA, THCA-A, and CBGA with the SARS-CoV-2 spike protein S1 C-terminal domain were modeled using AutoDock Vina (FIG. 2). FIG. 2 shows computational based modeling of the binding of cannabinoid acids to the SARS-CoV-2 spike protein S1 C-terminal domain using AutoDock Vina. The active site residues of the S1 subunit are shown in yellow. CBGA is predicted to bind to an allosteric site (−6.6 kcal/mol free energy of binding) (FIG. 2A). Although less favorable (−6.2 kcal/mol), CBGA can also bind to the orthosteric site on the S1 C-terminal domain (FIG. 2B). THCA-A (FIG. 2C) and CBDA (FIG. 2D) are predicted to bind at the orthosteric site with free energies of binding −6.5 kcal/mol and −6.3 kcal/mol, respectively.

In agreement with the MagMASS rank ordering of ligands (Table 3), the free energy of binding was greatest for CBGA (−6.6 kcal/mol) followed by THCA-A (−6.5 kcal/mol) and CBDA (−6.3 kcal/mol). The optimum binding mode for CBGA was at an allosteric site with a binding pocket dominated by hydrophobic residues within a van der Waals distance of 4 Å, namely Phe374, Leu368, Phe342, Trp436, Ala344, Leu441 (FIG. 2A). The hydrophobic isoprenyl group of CBGA interacted with the hydrophobic residues Leu368, Phe342, Phe374, and Trp436 while the pentyl group interacted with Ala344, Leu441 and amide of Thr345. The carboxylic acid group formed a hydrogen bond with the Asp343 amide side chain, while the hydroxyl groups at positions 1 and 5 formed hydrogen bonds with the side chains of Asp343 and Arg509, respectively. Although less favorable, CBGA was also predicted to bind orthosterically to the spike protein S1 C-terminal domain with −6.2 kcal/mol free energy of binding (FIG. 2B).

Unlike CBGA, CBDA and THCA-A were predicted to bind preferentially within the orthosteric site of the spike protein S1 subunit. The key interactions for CBDA include hydrogen bonding between carboxylic acid group and the Arg403 side chain and hydrophobic interactions between the CBDA aromatic ring and the Tyr495 side chain (FIG. 2C). Additional hydrophobic contributions were made by Tyr505, Gly496, and Tyr453. The hydroxyl group at position 5 of CBDA formed a hydrogen bond with the amide group of Gly496. THCA-A was predicted to bind at the surface of the orthosteric site in a hydrophobic region consisting of Tyr495, Phe497, Tyr505, Gly496 (FIG. 2D). Hydrogen bond interactions could form between the carboxylic acid and ASP501 as well as between the hydroxyl group at position 1 and the carbonyl group of Tyr505.

TABLE 3
MagMASS ranking of hemp cannabinoids for binding to the SARS-
CoV-2 spike protein (mean ± S.E. (N = 3).

| | UHPLC | |
|---|---|---|
| | retention | Fold peak area |
| Cannabinoid^{a} | time (min) | enrichment^{b} |
| Cannabigerolic acid (CBGA) | 3.8 | 20.5 ± 0.51 |
| Tetrahydrocannabinolic acid (THCA-A) | 8.2 | 16.7 ± 2.2 |
| Cannabidiolic acid (CBDA) | 3.7 | 12.2 ± 0.52 |
| Cannabinolic acid (CBNA) | 6.5 | 5.6 ± 1.4 |
| Cannabigerol (CBG) | 4.1 | 3.4 ± 0.82 |
| Cannabinol (CBN) | 5.7 | 3.4 ± 0.78 |
| Δ⁸-Tetrahydrocannabinol (Δ8-THC) | 6.8 | 3.1 ± 0.81 |
| Δ⁹-Tetrahydrocannabinol (Δ9-THC) | 6.8 | 3.0 ± 0.77 |
| Cannabidiol (CBD) | 4.2 | 2.9 ± 0.72 |
| Cannabichromene (CBC) | 8.1 | 2.9 ± 0.75 |
| Cannabidivarin (CBDV) | 3.0 | 1.6 ± 0.17 |

^{a}Equimolar cannabinoid mixture (0.10 μM) incubated with S1 subunit of spike protein (0.17 μM).
^{b}Fold peak area enrichment = (UHPLC-MS/MS peak area experiment)/(UHPLC-MS/MS peak area negative control using denatured spike protein S1 subunit).

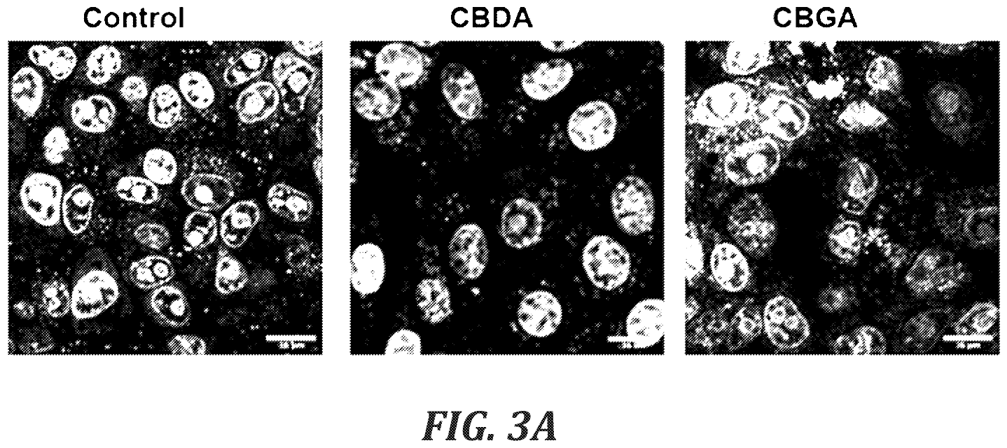

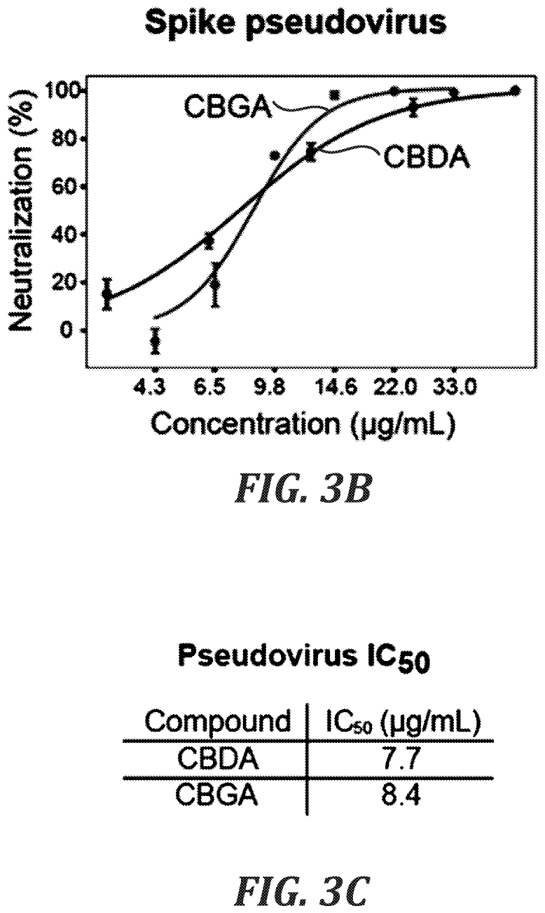

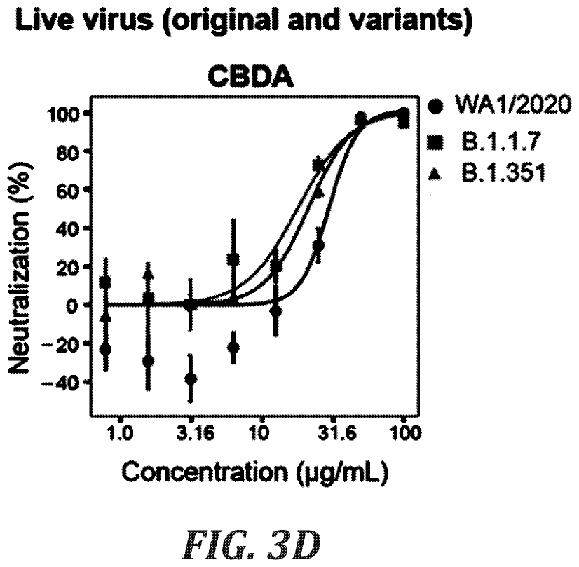

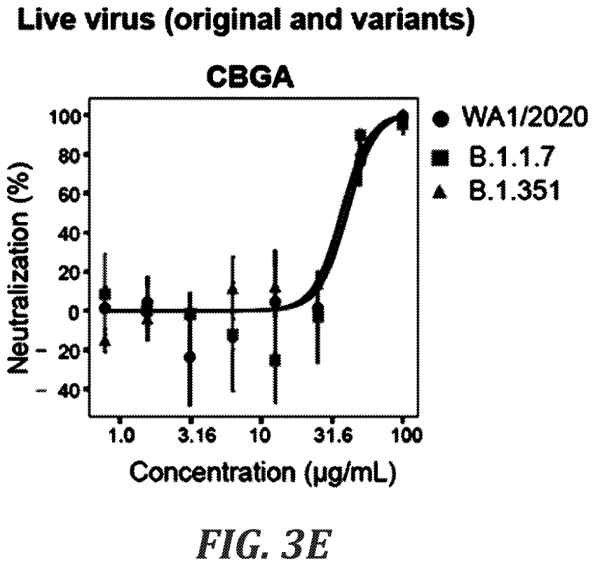

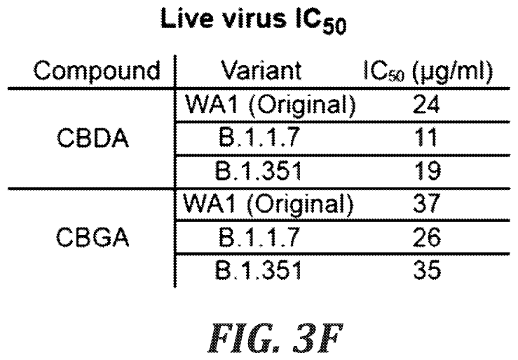

Inhibition of SARS-CoV-2 cell entry. To determine if CBDA or CBGA could prevent infection by blocking SARS-CoV-2 cell entry, pseudovirus and live SARS-CoV-2 virus cell infection assays were carried out. The live SARS-CoV-2 virus was incubated with 25 μg/mL of either CBDA, CBGA or vehicle control (DMSO) and then infected Vero E6 cells. After 24 h post infection, cells were stained with anti-double stranded RNA (dsRNA) antibody known to bind specifically to viral RNA. An absence of SARS-CoV-2 viral RNA was found in cells treated with either cannabinoid (FIG. 3A). To quantify the level of inhibition, spike protein pseudotyped lentiviral particles with a GFP reporter gene, and HEK 293T cells overexpressing ACE-2 were infected for 48 h with these lentiviral particles following treatment with varying concentrations of CBDA, CBGA, or vehicle control. The number of infected cells was quantified by fluorescence microscopy, and the concentration which reduced pseudovirus infections by half (IC₅₀) was 7.7 μg/mL for CBDA and 8.4 μg/mL for CBGA (FIG. 3B-C).

FIG. 3 shows CBD compounds block viral entry of SARS-CoV-2 through spike binding. Neutralization of spike protein pseudotyped lentivirus and multiple variants of live SARS-CoV-2 virus by cannabinoids CBDA and CBGA. Representative images of high resolution microscopy of SARS-CoV-2 (WA1/2020) infected Vero E6 cells treated with 25 μg/mL CBDA, CBGA, or vehicle (control) (FIG. 3A). Cells were stained with anti-ds-RNA (red) antibody to visualize replication sites formed during infection. DAPI (blue) was used to stain nuclei. Infection of ACE2 293T cells with SARS-CoV-2 spike pseudotyped lentivirus in the presence of CBDA or CBGA (FIG. 3B). Percent neutralization was determined by quantification of total GFP signal resulting from successful pseudovirus infection, normalized to vehicle control (n=3). Table of IC₅₀ values for pseudovirus experiments (FIG. 3C). Live-virus infection of Vero E6 cells with SARS-CoV-2 variants (WA1/2020, B.1.1.7, and B.1.351) in the presence of CBDA (FIG. 3D) or CBGA (FIG. 3E). Percent neutralization was normalized to vehicle control wells (n=3). Table of IC₅₀ values for live-virus experiments shown in FIGS. 3D and 3E (FIG. 3F). IC₅₀ values were determined by fitting data to a three-parameter model for pseudotype infection (FIG. 3C) and live-infection (FIG. 3F) experiments.

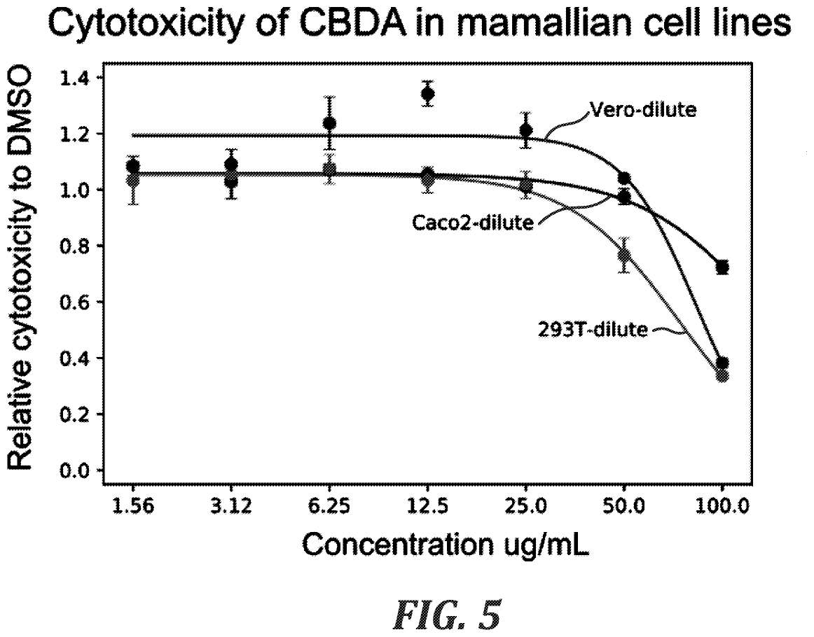

The cytotoxicity of these compounds was insignificant below 50 μg/mL for Caco2, 293T-ACE2, and Vero cells (FIG. 5). CBDA in mammalian cell lines at concentrations used for neutralization. Cytotoxicity of CBDA was analyzed at concentrations used for neutralization to disentangle inhibition of viral entry and loss of cell fitness. Fluorescent resazurin assay was used to estimate cell viability for each cell line in presence of CBDA dilutions. Fluorescent signal was normalized to DMSO-only control for each cell line. Data are presented from two independent experiments.

To validate the virus neutralizing capabilities of CBDA and CBGA, focus forming assays were performed using authentic SARS-CoV-2 virus (Isolate USA-WA1/2020). Vero E6 cells were utilized for these experiments due to their high susceptibility to the virus and common use in SARS-CoV-2 live-virus studies. Focus forming assays were performed using serial dilutions of CBDA or CBGA which were incubated with infectious SARS-CoV-2 for 1 h prior to infection. As in the pseudovirus neutralization assay, CBDA and CBGA prevented SARS-CoV-2 entry into Vero E6 cells with IC₅₀ values of 24 μg/mL and 37 μg/mL (FIGS. 3D-3F), respectively.

Emerging variants of concern (VOC), including B.1.1.7 and B.1.351, have been shown to resist neutralization by antibodies generated against earlier lineages of SARS-CoV-2. To assess whether blockage of cell entry by CBDA and CBGA is variant dependent, additional focus forming assays were performed using the live SARS-CoV-2 variants B.1.1.7, containing the N501Y spike protein mutation, and B.1.351, containing the K417N, E484K, and N501Y spike mutations. Like WA1/2020 infections, CBDA and CBGA both blocked B.1.1.7 infection with IC₅₀ values of 11 μg/mL and 26 μg/mL respectively. B.1.351 was neutralized as well by both compounds with IC₅₀ values of 19 μg/mL and 37 μg/mL, respectively (FIG. 3D-F), indicating no substantial loss of activity against these VOCs.

### Results

Originally invented for high-throughput screening of pools of combinatorial libraries, the selectivity, sensitivity, and speed of AS-MS approaches like MagMASS are also ideal for screening natural products mixtures such as botanical extracts. Compared with conventional high-throughput screening utilizing fluorescence or absorbance readouts (such as FRET or fluorescence polarization), AS-MS offers advantages such as compatibility with any type of ligand mixture, matrix, and assay buffer and no requirement for fluorescent tags. Uniquely, AS-MS does not suffer from interference from samples containing fluorophores or chromophores, which are common in natural products. One of the newer AS-MS techniques, MagMASS offers advantages compared with the other AS-MS approaches that use ultrafiltration or size exclusion such as case of automation and faster separation of receptor-ligand complexes from unbound compounds, which minimizes ligand loss due to premature dissociation from the receptor and maximizes sensitivity. Therefore, MagMASS is an ideal platform for the discovery of natural ligands of the SARS-CoV-2 spike protein.

Recently, the crystal structure of C-terminal domain of the SARS-CoV-2 spike protein in complex with the human ACE2 receptor was solved. The key interactions involve residues along the spike protein C-terminal domain interface that contribute to a network of hydrogen bonding and salt-bridge interactions with the ACE2 receptor. The residues on the spike protein involved in the binding to ACE2 include A475, N487, E484, Y453, K417, G446, Y449, G496, Q498, T500, G502, Y489, and F486. The interaction of the virus to the ACE2 receptor is mainly contributed by the polar interactions resulting from hydrophilic residues on the surface of the spike protein C-terminal domain.

In the AutoDock Vina docking program, the ligand docking in the active site is based on algorithms that take into consideration the steric, hydrophobic bonding, and hydrogen bonding interactions between the ligand and active site residues. The best predicted binding conformation should have the lowest free energy of binding (kcal/mol). CBGA gave the lowest free energy of binding (−6.7 kcal/mol) to an allosteric site, with a root mean square deviation of 24.3 from the orthosteric site. On the other hand, THCA-A and CBDA had slightly higher energies of binding at −6.5 and −6.3 kcal/mol, respectively. Overall, the MagMASS data show that CBGA binds to the spike protein S1 subunit strongly in cannabinoid mixtures, suggesting that it binds allosterically and does not compete for binding with orthosteric cannabinoid ligands.

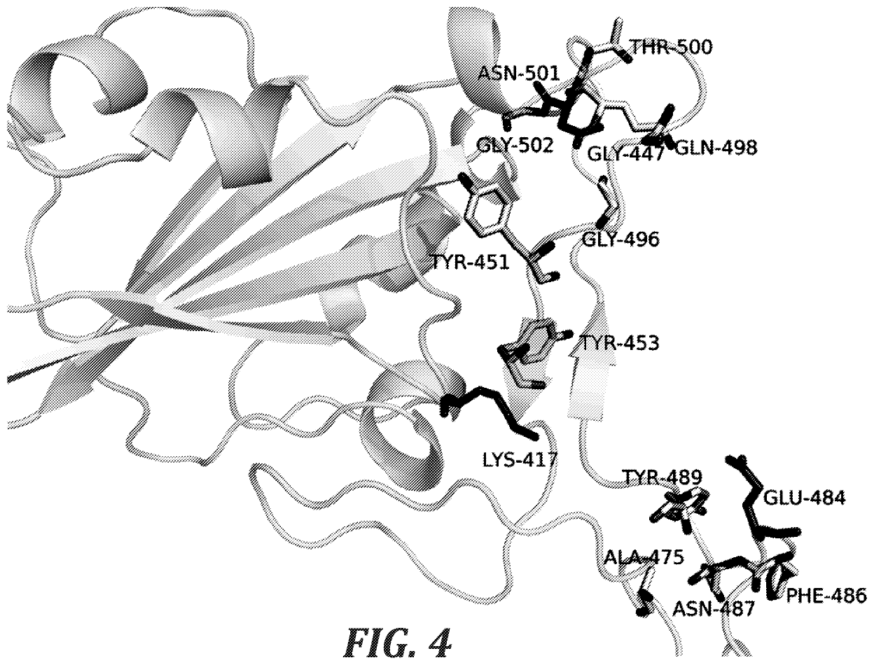

Variants of the SARS-CoV-2 virus such as B.1.1.7 and B.1.351 include amino acids in the spike protein S1 subunit that interact with the ACE2 receptor. For example, the N501Y mutation was identified by bioinformatics analysis of data derived by metagenomics sequencing of samples obtained from a patient with persistent SARS-CoV-2 infection. Other highly infectious variants identified that include mutation of the active site residues include N501T, K417 and E484K (FIG. 4). With the rapid mutations occurring, a novel inhibitor capable of binding to an orthosteric site would be of great interest in the intervention of SARS-CoV-2 variants characterized by active site mutations.

The described infection inhibition assay results clearly indicate that CBDA and CBGA are both able to block cell entry by SARS-CoV-2. The concentrations needed to block infection by 50% of viruses is high but might be clinically achievable. For example, CBDA administered orally to human volunteers at 0.063 mg/kg showed greater bioavailability than CBD and produced maximum plasma concentrations of 0.21 μM. In beagle dogs, oral administration of CBDA at 1 mg/kg was well tolerated, 2-fold more bioavailable than CBD, and produced serum levels up to 1.42 μM. Although no data on the bioavailability of CBGA are yet available, the data for CBDA suggest that μM plasma and serum concentrations for CBGA should also be possible.

Previous reports have indicated that one possible mechanism for inhibition of SARS-CoV-2 by decarboxylated cannabidiol (CBD) is activation of innate immune mechanisms. However, the live-virus data indicate that inhibition by CBDA and CBGA occur at the point of cell entry. These mechanisms are not mutually exclusive and it remains possible that multiple cannabinoids in complex mixtures from plant extracts may act independently to inhibit SARS-CoV-2, potentially leading to enhanced effectiveness when compared to individual compounds.

One of the primary concerns in the ongoing pandemic is the spread of viral variants, of which there are many, with some of the most concerning and widespread being B.1.1.7 and B.1.351. These variants are well known for evading antibodies against early lineage SARS-CoV-2, which is particularly concerning due to the fact that current vaccination strategies rely on the early lineage spike RBD as an antigen. The data show minimal impact of the variant lineages on the effectiveness of CBDA and CBGA, a trend which will hopefully extend to other existing and future variants. Because it is believed that the primary binding site for CBGA is allosteric, there may even be reduced evolutionary pressure for SARS-CoV-2 to mutate their binding sites compared to the orthosteric binding sites typically favored by neutralizing antibodies. With widespread use of cannabinoids, resistant variants could still arise, but the combination of vaccination and CBDA/CBGA treatment should create a more challenging environment with which SARS-CoV-2 must contend, reducing the likelihood of escape.

### Materials and Methods

Affinity selection-mass spectrometry. Recombinant SARS-CoV-2 spike protein (RayBiotech; Peachtree Corners, GA) (˜72 kDa) containing an N-terminal His-tag was immobilized on Ni²⁺-nitrilotriacetic acid derivatized magnetic microbeads (AvanBio; Parsippany, NJ) for use in the affinity selection-mass spectrometry approach MagMASS. As a negative control, denatured spike protein was immobilized on identical magnetic microbeads. Positive control incubations used SBP-1 (RayBiotech), a 23 amino acid peptide with the sequence of IEEQAKTFLDKFNHEAEDLFYQS (SEQ ID NO: 2), which is identical to the ACE2 al helix sequence recognized by the SARS-CoV-2 spike protein. SBP-1 (33 nM) was incubated for 60 min with magnetic microbeads containing 50 pmol of immobilized active or denatured S1 protein in 300 μL binding buffer. After washing twice with 500 μL of 30 mM ammonium acetate to remove unbound ligand while the beads were retained by a magnetic field, ligand was released from the beads using 90% methanol in water (200 μL) and analyzed using UHPLC-LC/MS.

Extracts of hemp and isolates of specific cannabinoids were obtained from Klersun (Portland, OR), PharmEx (Salem, OR), and Columbia Hemp Trading Company (Corvallis, OR). Certified cannabinoid standards were purchased from Cayman Chemical (Ann Arbor, MI). Extracts (10 μg), mixtures of cannabinoid standards (0.10 μM each), or cannabinoid standards (0.10 μM) were incubated with 50 pmol of immobilized SARS-CoV-2 spike protein and screened using MagMASS as described above. The released ligand was analyzed using UHPLC-LC/MS on a Shimadzu (Kyoto, Japan) Nexera UHPLC system interfaced with an LCMS-9030 Q-ToF hybrid high resolution mass spectrometer or an LCMS-8050 triple quadrupole mass spectrometer. Reversed phase UHPLC separation was carried out using a Waters (Milford, MA) Acquity UPLC BEH C₁₈ column (1.7 μm, 130 Å. 2.1 mm×50 mm) with a 5 min linear gradient from 20% to 80% acetonitrile in 0.1% aqueous formic acid at a flow rate of 0.3 mL/min for the analysis of SBP-1 peptide. Cannabinoid separations were similar except that a 100 mm Waters Acquity UHPLC BEH C₁₈ column was used with a 1 min gradient from 50% to 75% acetonitrile followed by an 11 min gradient to 80% acetonitrile. The column was equilibrated to initial conditions for 1 min between analyses. Ligands eluting from the column were detected using positive ion or negative ion electrospray mass spectrometry. Natural product ligands for which structures have been reported in the literature were identified based on their elemental compositions determined using high resolution accurate mass measurements, tandem mass spectra, and comparison with authentic standards.

Equilibrium dissociation constants. The affinity constants for the binding of active compounds to the spike protein S1 subunit were determined by rapid equilibrium dialysis. First, the optimum time for full equilibration of the RED device obtained from ThermoFisher (Waltham, MA) was determined with each of the compounds by adding 1 μM spike protein in buffer (300 μL) to the protein chamber and adding blank phosphate buffered saline, pH 7.2 (500 μL) to the buffer chamber. CBGA or CBDA was spiked into the protein chamber at a final concentration of 2.5 μM. During incubation at 37° C. on an orbital shaker at 200 rpm, aliquots (30 μL) were sampled from the sample and buffer chambers at 0.5, 1, 2, 3, 4, 5, 6 and 7 h and mixed with equal volumes of buffer, 300 μL of ice-cold 90% aqueous acetonitrile containing 0.1% formic acid, and 500 ng/ml d4-daidzein (internal standard), vortex mixed and incubated on ice for 1 h. After centrifugation at 18,000 g for 30 min, the supernatant was removed and ligand concentration was measured using UHPLC-MS/MS. Next, the equilibrium dissociation constants of CBGA and CBDA were determined by incubating the spike protein S1 subunit with different concentrations of the ligands ranging from 0.05-500 μM in triplicate. After 5 h for CBGA or 4 h for CBDA, the concentrations of each ligand in the sample and buffer chambers were measured using UHPLC-MS/MS as described above. Data analysis and fitting was carried out using Microsoft Excel (Seattle, WA) and KaleidaGraph v4.1 (Reading, PA).

Ligand docking. The computational aided modeling of cannabinoids was carried out using AutoDock Vina (Scripps Research Institute, La Jolla, CA). The coordinates of the crystal structure of SARS-CoV-2 spike protein C-terminal domain were downloaded from the Protein Data Bank (PDB, ID number 6LZG). The ChemDraw structures of the ligands were converted to .pdb files using Pymol. The protein data were loaded into the AutoDock Vina program, and the search space was defined around the known orthosteric site and the file was converted to .pdbqt. Similarly, the ligands were individually loaded and converted to .pdbqt files.

Pseudotyped lentivirus production. Pseudovirus was prepared as previously described. 293T cells, seeded one day ahead with 2 million cells in 6 cm TC-treated dishes, were transfected with lentivirus packaging plasmids, SARS-CoV-2 S plasmid, and IzGreen reporter plasmid. After transfection, cells were incubated at 37° C. for 60 h. Viral media were filtered with a 0.45 μm syringe filter and snap frozen in liquid nitrogen before storing at −80° C. Virus stocks were titrated on 293T-ACE2 cells treated with 50 μL of 5 μg/mL polybrene (Sigma-Aldrich; St. Louis, MO). Titer was determined by fluorescence microscopy using a BZ-X700 all-in-one fluorescent microscope (Keyence; Itasca, IL).

SARS-CoV-2 virus propagation. SARS-CoV-2 isolates USA/CA_CDC_5574/2020 [lineage B.1.1.7] (NR-54011), hCoV-19/South Africa/KRISP-K005325/2020 [lineage B.1.351] (NR-54009), and USA-WA1/2020 1 [lineage A] (NR-52281) were obtained through BEI Resources, diluted 1:10, and added onto 70% confluent Vero E6 cells. The cells were incubated for 1 h at 37° C. with rocking every 15 min. Additional media were added according to the manufacturer's recommended culture volume, and the cells were incubated for 72 h in a tissue culture incubator. Supernatant was centrifuged at 3,000×g for 5 min before aliquoting and freezing at −80° C.

Pseudovirus neutralization assay. Pseudovirus neutralization was performed as previously described. Briefly, 293T-ACE2 cells were seeded at 10,000 cells per well on tissue culture treated, poly-lysine treated 96-well plates. Cells were grown overnight at 37° C. LzGreen SARS-CoV-2 S pseudotyped lentivirus was combined with two-fold serial dilutions of CBDA and CBGA in DMSO or vehicle control. Virus-drug mixture was incubated at 37° C. for 1 h after which virus was added to 293T-ACE2 treated along with 5 μg/mL polybrene. Cells were incubated with neutralized virus for 44 h, then fixed with 4% formaldehyde for 1 h at room temperature, incubated with DAPI for 10 min at room temperature, and imaged with a BZ-X700 all-in-one fluorescent microscope (Keyence; Itasca, IL). Total area of DAPI and GFP fluorescent signal were calculated using included microscope software (Keyence). To account for variability in cell count, green fluorescent signal was normalized to DAPI signal. For conditions with fewer DAPI foci, the modal value of DAPI signal for each set of replicates was used for normalization across that condition. Otherwise DMSO control values were used in normalization to manage DAPI inconsistency across replicates. IC₅₀ values were calculated with combined replicate data in python using a 3-parameter logistic model and plotted with the matplotlib data visualization library.

Focus forming assay for live SARS-CoV-2. Focus forming assays were performed as previously described. In brief, 96-well plates with subconfluent Vero E6 cells were infected with 50-100 virus titer per well of the original SARS-CoV-2 strain (WA-1/2020) or the variants (B.1.1.7, or B.1.351) in buffer containing CBDA or CBGA ranging from 100 μg/mL to 0.625 μg/mL. DMSO was used as a vehicle control. The virus and drug mixtures were incubated for 1 h at 37° C. prior to addition to cells. The mixture was incubated with cells for 1 h at 37° C. before addition of overlay media (Opti-MEM, 2% FBS, 2% methylcellulose). Infection was allowed to proceed for 48 h, then plates were fixed for 1 h in 4% formaldehyde in phosphate buffered saline (PBS). Cells were permeabilized (PBS, 0.1% saponin, 0.1% bovine serum albumin) for 30 min. Anti-SARS-CoV-2 spike protein alpaca immune serum was diluted 1:5,000 in permeabilization buffer and incubated on plates overnight at 4° C. Plates were washed three times with PBS with 0.1% Tween-20 (wash buffer) and incubated with anti-llama-HRP at 1:20,000 for 1 h at room temperature. Following three more washes in wash buffer, plates were developed with TrueBlue (seracare) for 30 min before being imaged (CTL immunospot) and counted (Viridot). Three separate dilution series were prepared for each experiment, each of which was used to prepare three technical replicates. IC₅₀ values were calculated with combined replicate data in python using a 3-parameter logistic model and plotted with the matplotlib data visualization library.

Immunofluorescence. Vero E6 cells were seeded on 96-well glass bottom optical plates coated with poly-lysine solution; 20,000 cells were seeded per well. Cells were infected with SARS-CoV-2 as described above. At 24 h post-infection, cells were fixed with 4% PFA in PBS for 1 h. 96-well plate with SARS-CoV-2 infected Vero E6 cells were permeabilized with 2% BSA, 0.1% Triton-X-100 in PBS. Transfected cells were incubated for 2 h at room temperature with a mouse anti-dsRNA antibody (Millipore Sigma) to stain SARS-CoV-2 replication sites in infected cells. Anti-mouse IgG AF555, conjugated secondary antibodies were added at 1:500 dilution for 1 h at RT (Invitrogen; Carlsbad, CA). Confocal imaging was performed with a Zeiss LSM 980 using a 63× Plan-Achromatic 1.4 NA oil immersion objective. Images were processed with Zeiss Zen Blue software. Maximum intensity z-projections were prepared in Fiji.

### Example 2

### Methylated Cannabinoid Acids

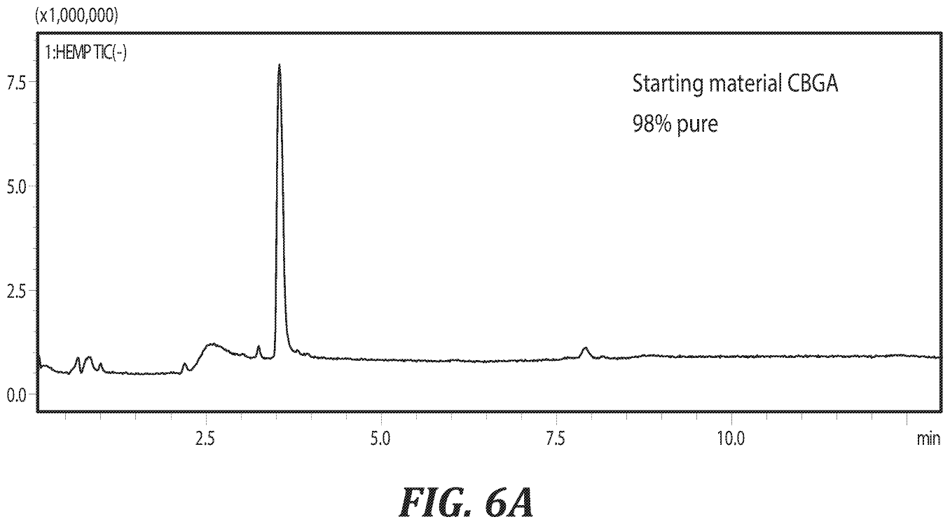

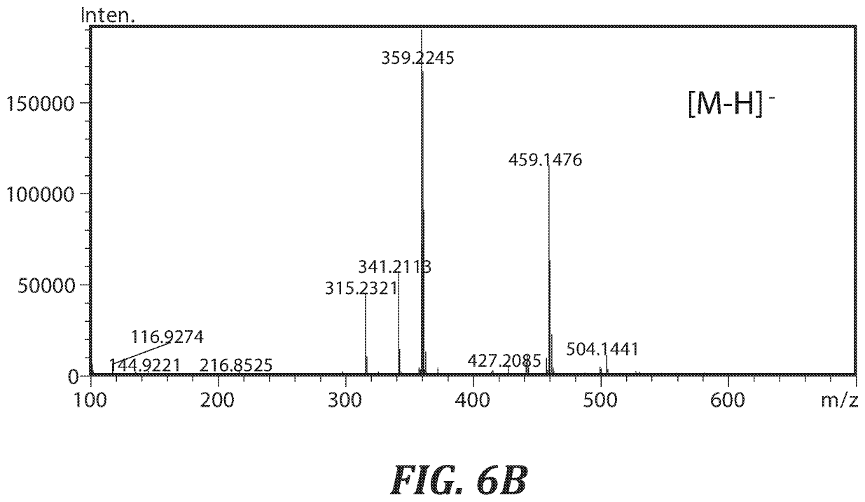

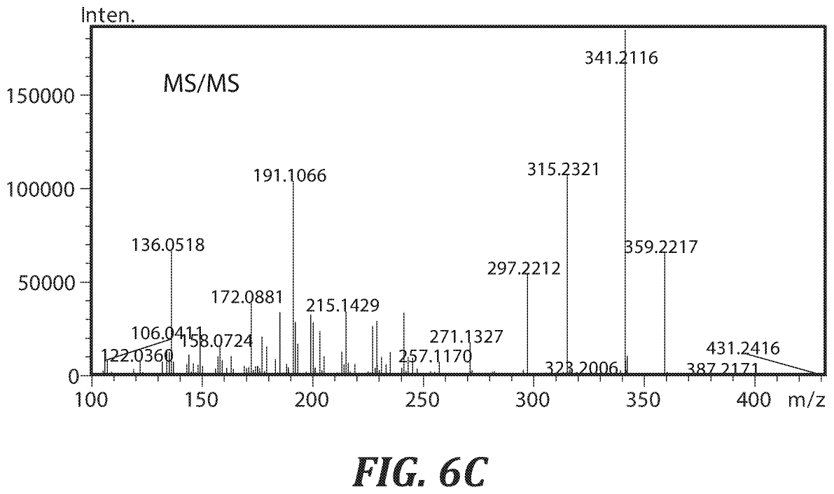

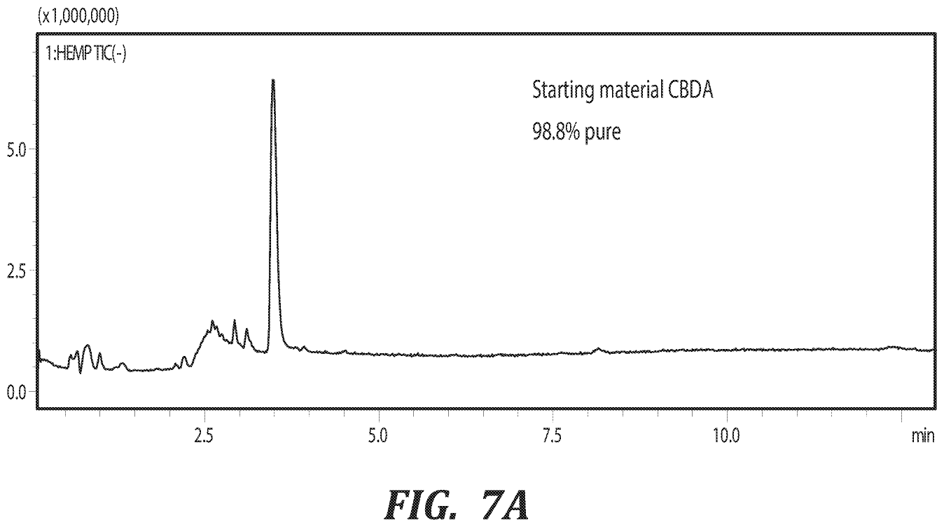

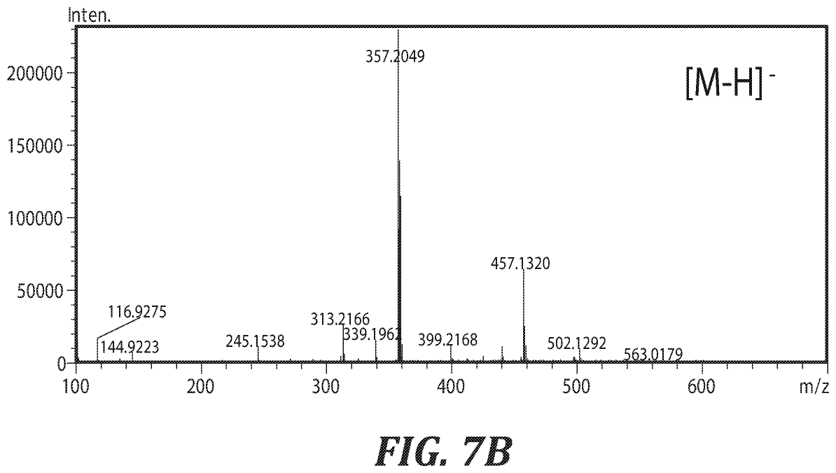

CBGA (FIG. 6) and CBDA (FIG. 7) methylated using TMS-diazomethane in the protocol described in Kühnel, et al., Angewandte Chemie International Edition 2007, 46, 7075, and illustrated below.

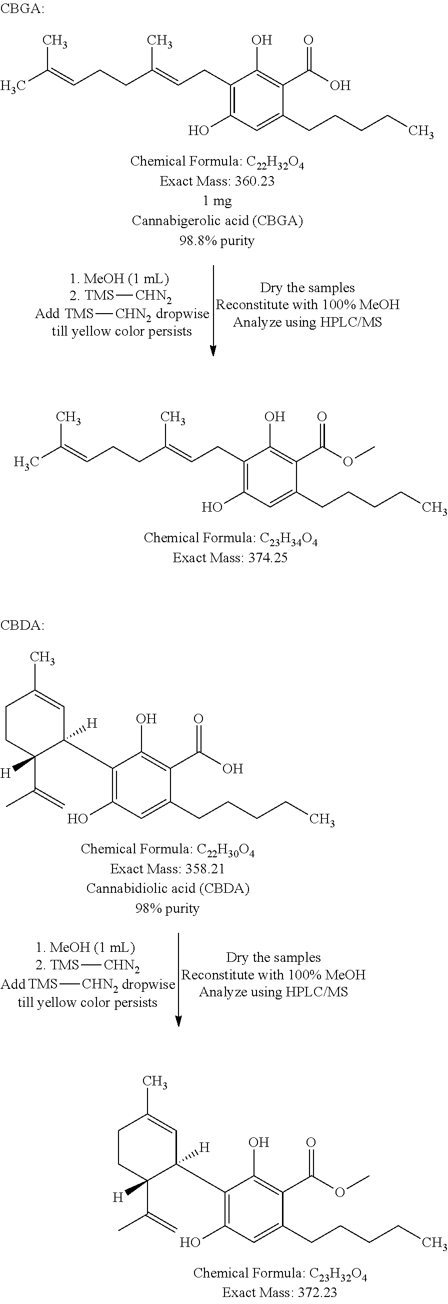

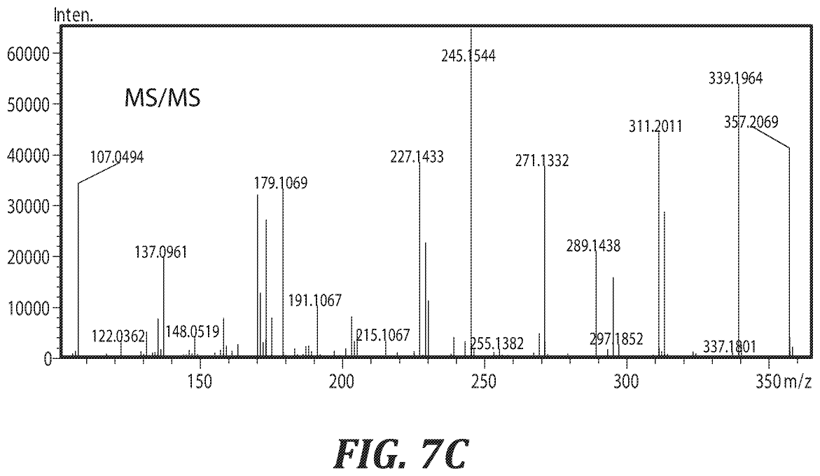

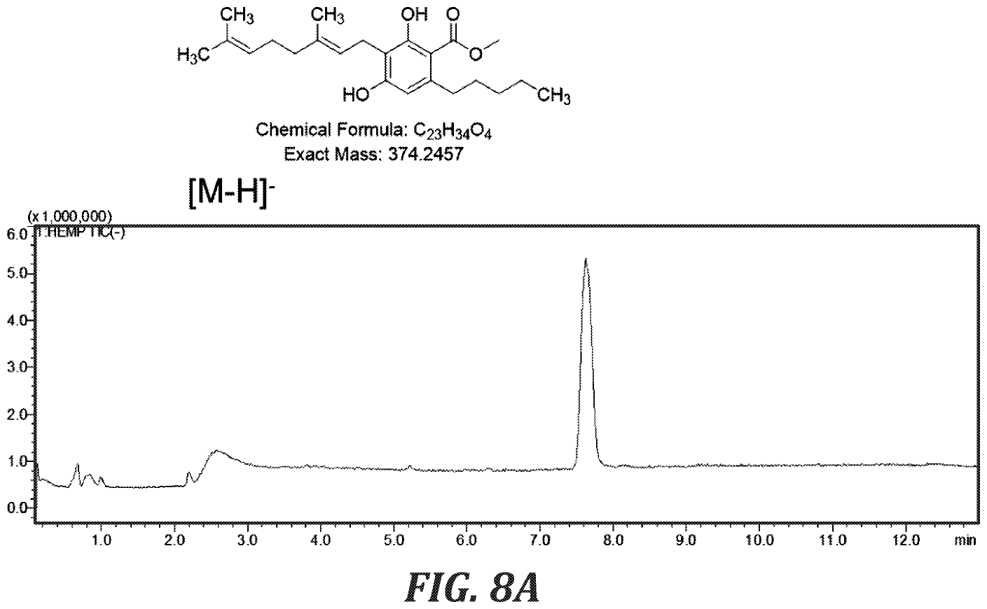

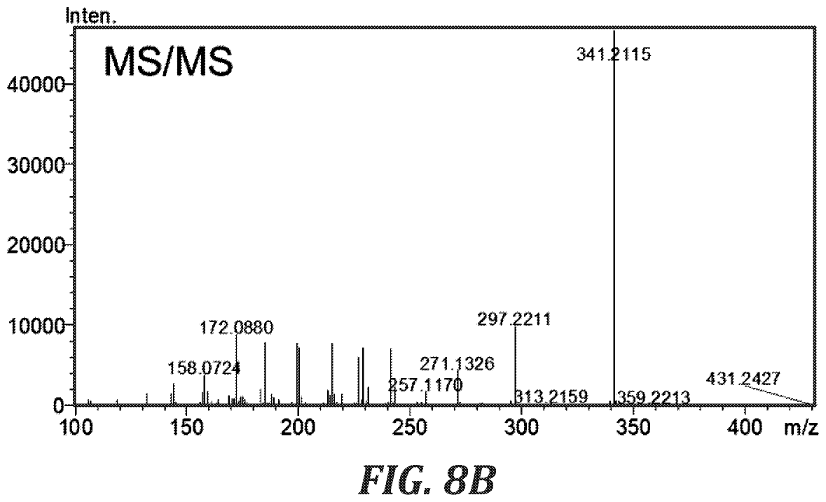

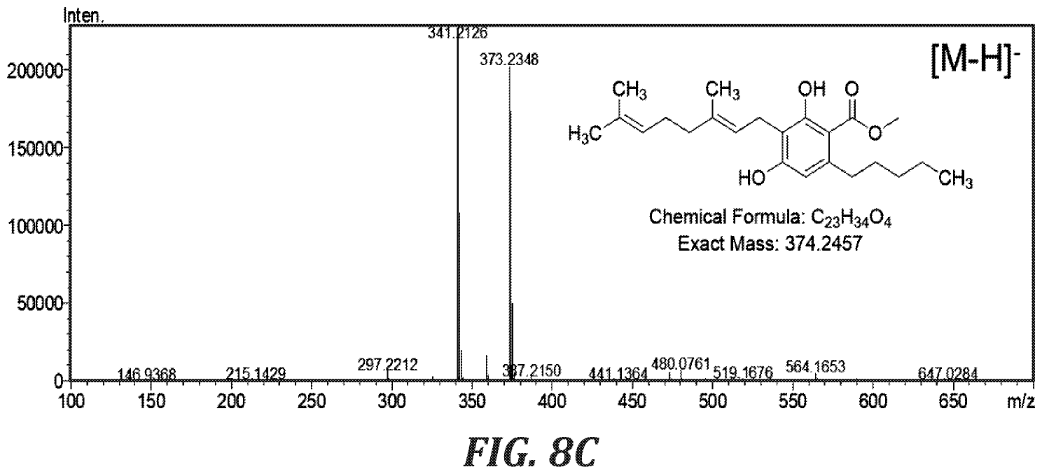

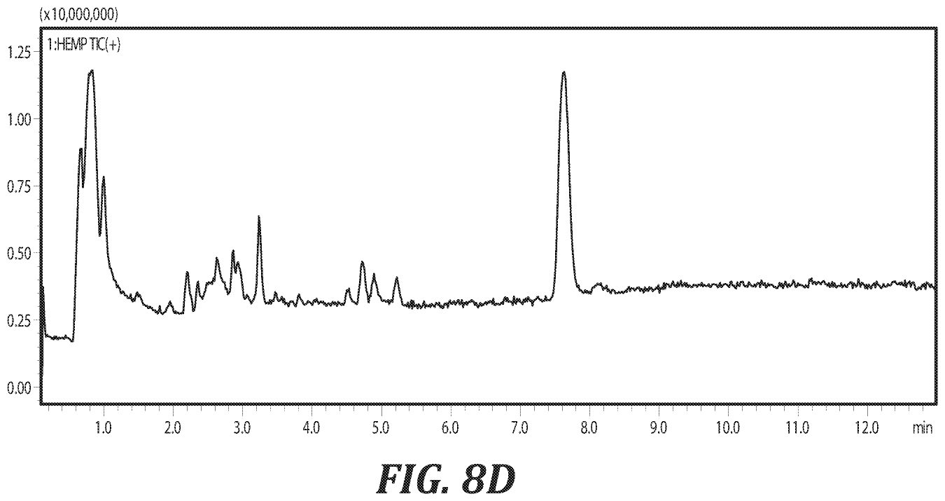

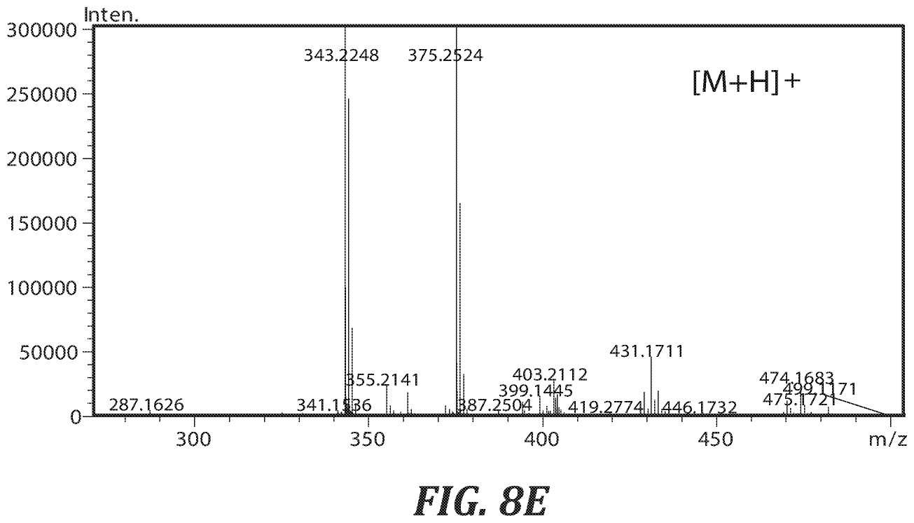

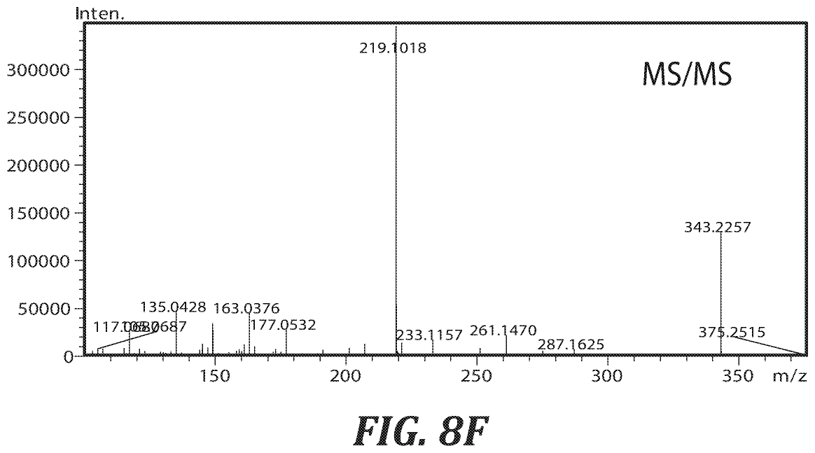

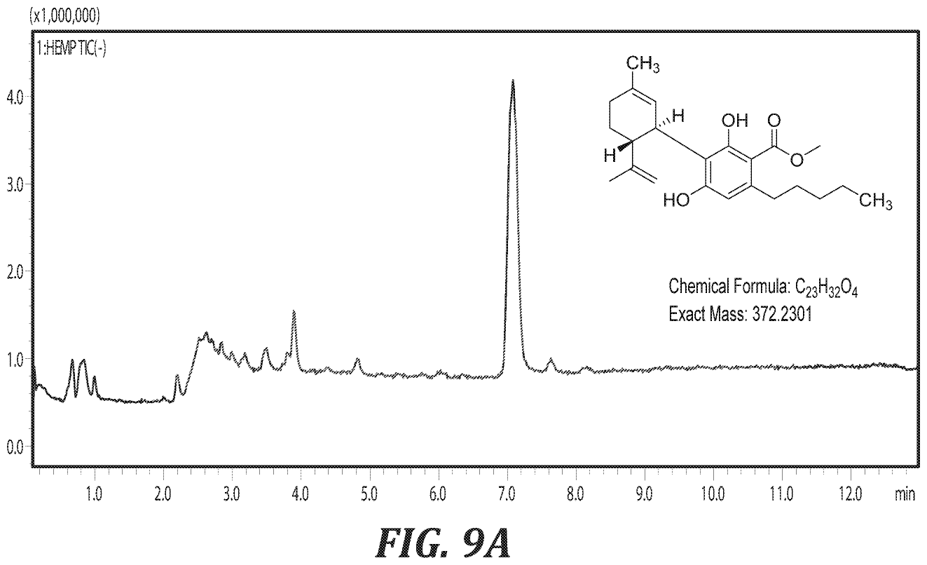

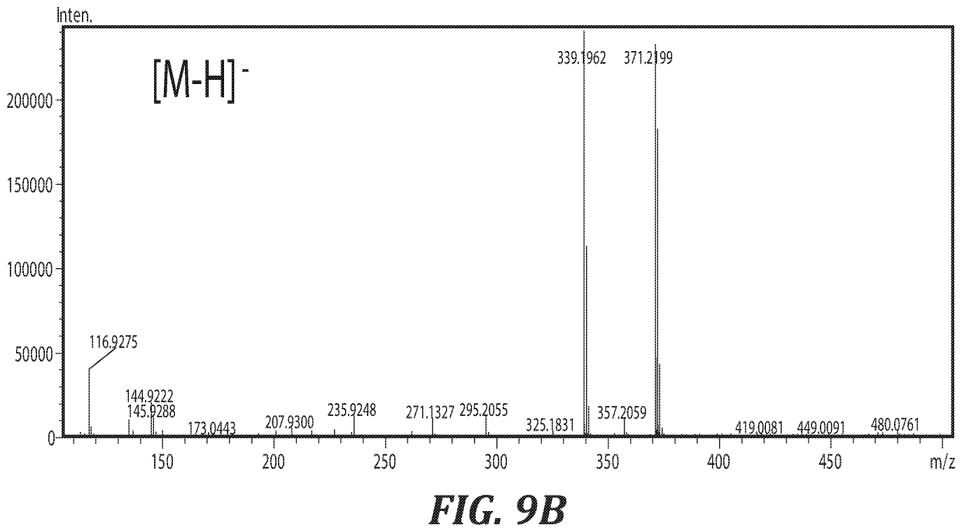

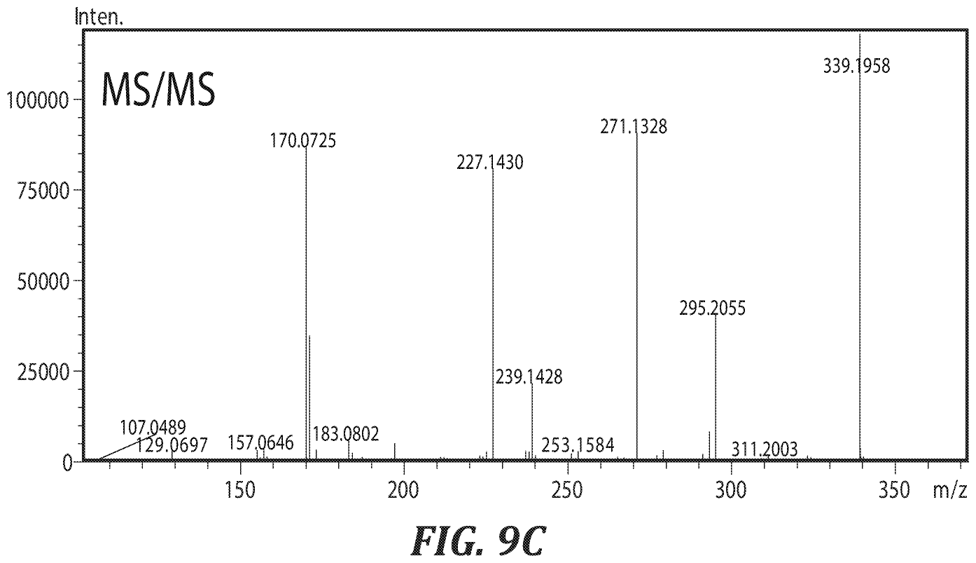

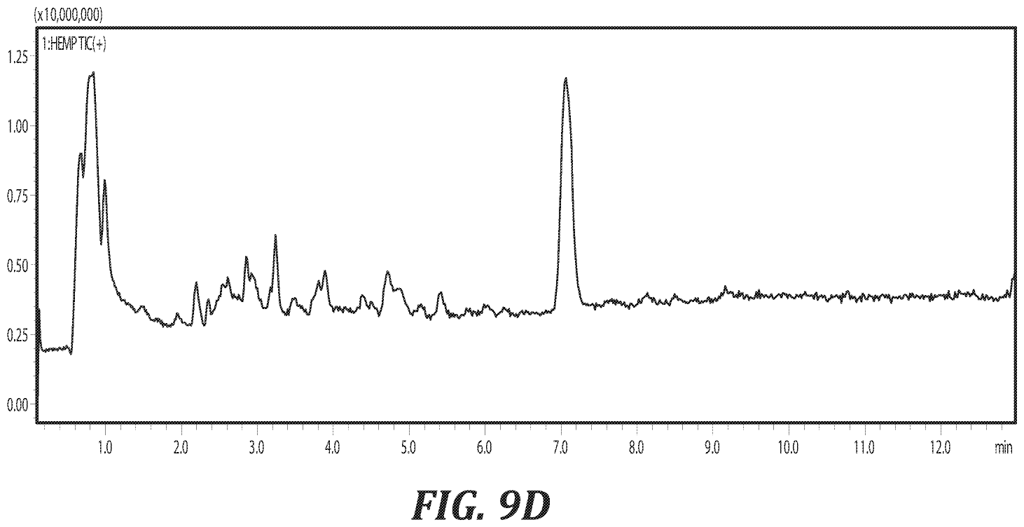

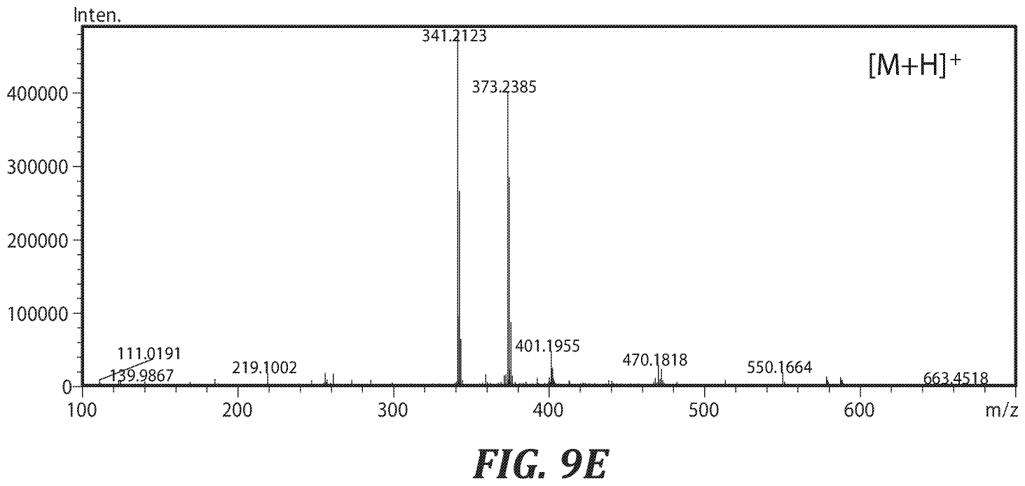

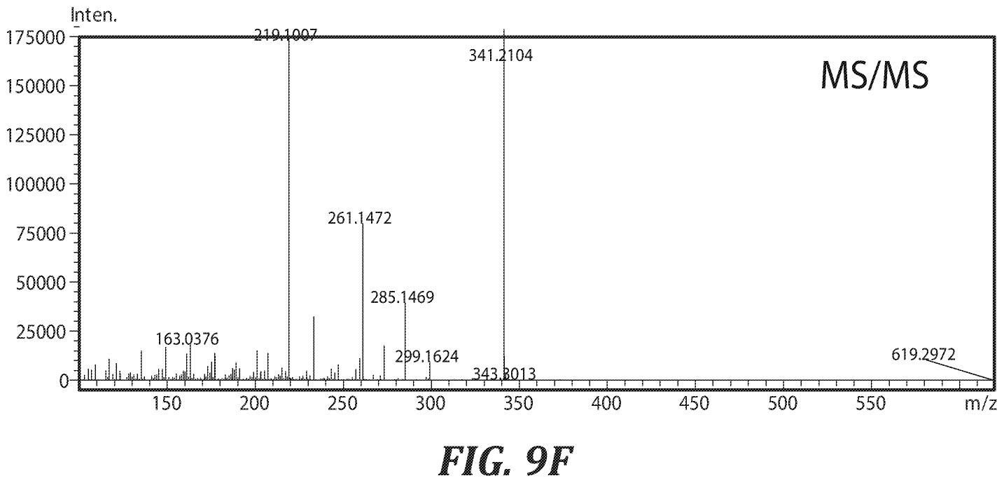

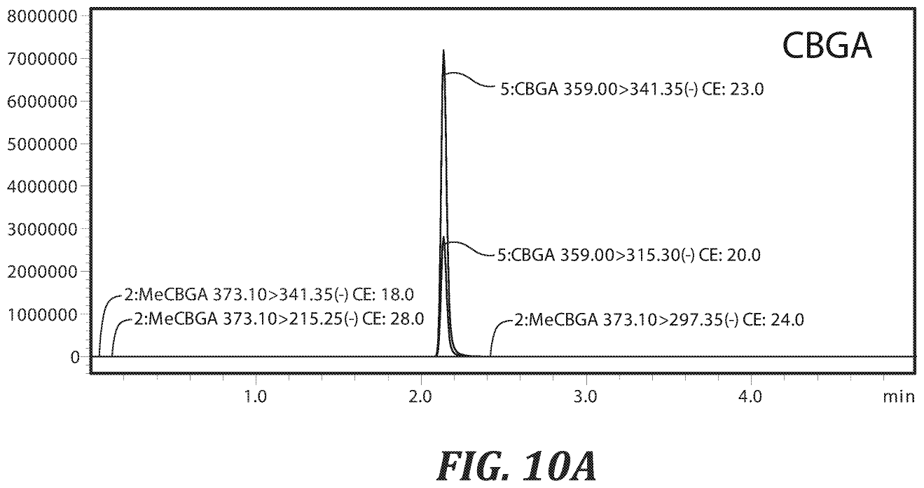

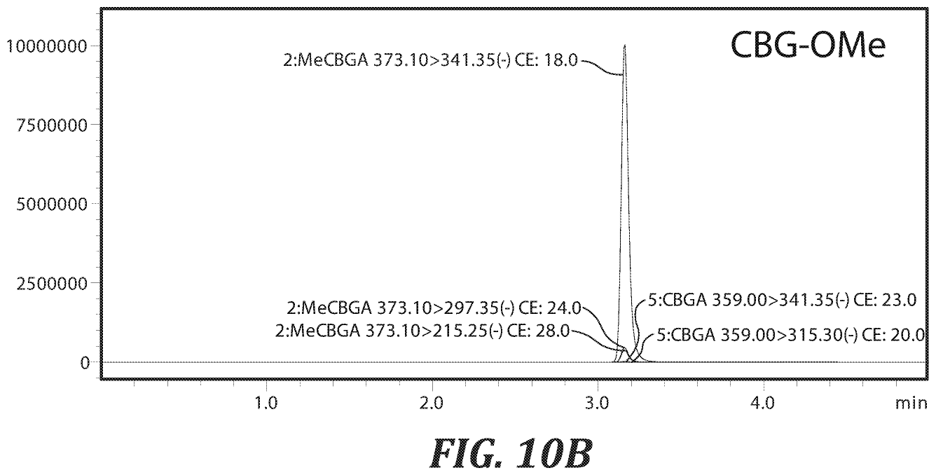

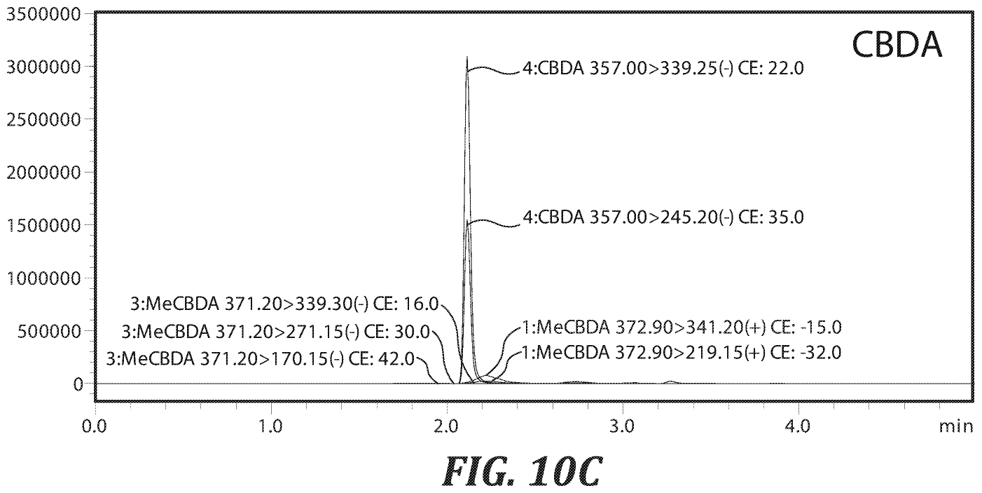

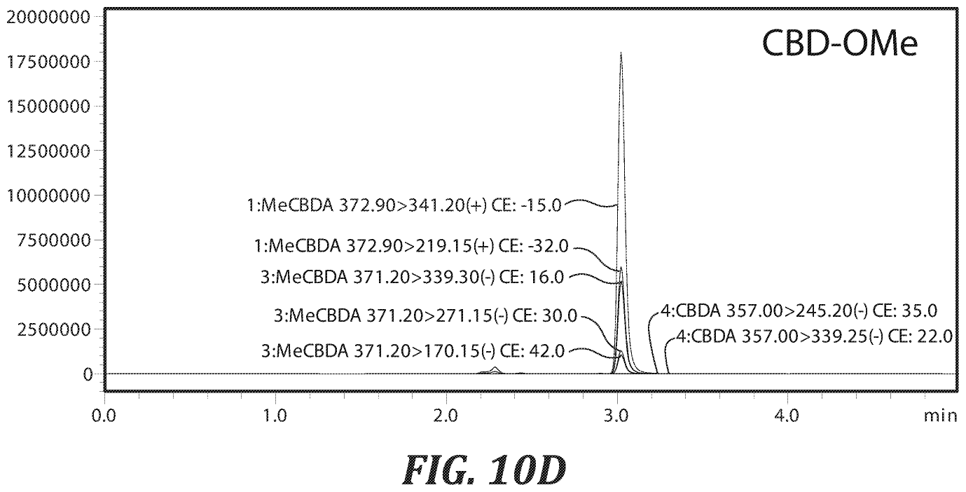

The HRMS scan spectra of the products are shown in FIG. 8 (MeCBGA) and FIG. 9 (MeCBDA). It was established that no starting material was observed in the methyl ester products synthesized from CBGA (FIG. 10A) and CBDA (FIG. 10B).

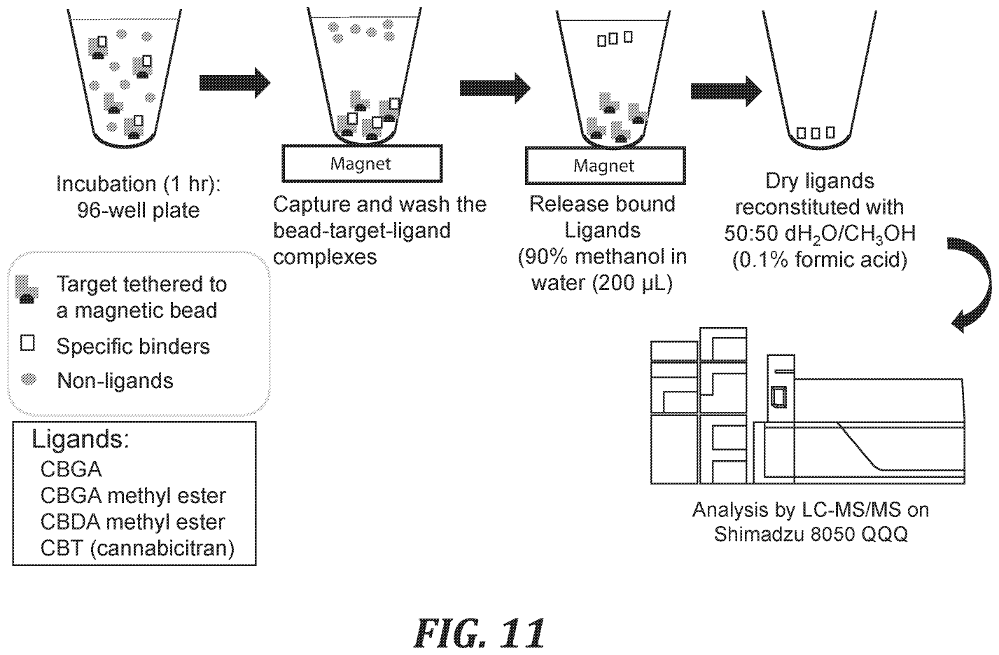

The resulting MeCBGA and MeCBDA were assessed using a protocol similar to the one described in Example 3 and as illustrated in FIG. 11. A summary of the reagents used is shown in Table 4.

TABLE 4
Reagents.

| | Assay 1: Intact | | Control 1: Denatured | |
|---|---|---|---|---|
| Reagents (Stock) | S1 | | S1 | |
| Ligand (150 μg/mL) | 1 μg/mL | (1.0 μL) | 1 μg/mL | (1.0 μL) |
| Spike (13600 pM) | 200 pmol | (2.5 μL) | 200 pmol | (2.5 μL) |

| | | |
|---|---|---|
| Ni-NTA beads (50 mg/mL) | 8 μL | 8 μL |
| Phos. Buffer (pH 7.2)-μL | 138.5 | 138.5 |
| Total volume (μL) | 150 | 150 |

The activity of the methylated compounds was tested using the methods described in Example 1. The results are shown in Table 5.

TABLE 5
Activity of the methylated compounds.

| | Fold Peak Area Enrichment | | |
|---|---|---|---|
| Phytocannabinoid | Standards mixture | Extract #5 | Pure compound |
| CBGA | 20.5 ± 0.51 | | 10.9 ± 1.3 |
| THCA-A | 16.7 ± 2.2 | | 19.7 ± 2.0 |
| CBDA | 12.2 ± 0.52 | 15.0 ± 0.5 | 15.5 ± 1.1 |
| CBG | 3.4 ± 0.82 | | |
| CBN | 3.4 ± 0.78 | | |
| Δ⁸-THC | 3.1 ± 0.81 | | |
| Δ⁹-THC | 3.0 ± 0.77 | | |
| CBD | 2.9 ± 0.72 | | |
| CBC | 2.9 ± 0.75 | | |
| CBDV | 1.6 ± 0.17 | | |
| CBG-OMe | | | 1.8 ± 0.07 |
| CBD-OMe | | | 1.5 ± 0.12 |

Additionally, a cannabinoid mixture of A9-THC, A8-THC, CBG, THCA-A, CBD, CBC, CBN, CBGA, CBDA, and CBDV was tested. CBGA binds to Spike S1 better when in the mixture compared to its pure form. Loss of activity of methylated CBGA and CBDA and low activity of CBG and CBD suggests that COOH group is important for binding activity of the cannabinoid hits identified.

### Example 3

### Magnetic Microbead Affinity Selection Screening

Affinity selection-mass spectrometry, which includes magnetic microbead affinity selection-screening (MagMASS), is useful for the discovery of ligands in complex mixtures that bind to pharmacological targets. Therapeutic agents are needed to prevent or treat COVID-19, which is caused by the severe acute respiratory syndrome coronavirus 2 (SARS-CoV-2). The first step in the infection of human cells by SARS-CoV-2 is binding of the virus spike protein subunit 1 (S1) to the human cell receptor angiotensin converting enzyme-2 (ACE2). Like antibodies, small molecules also have the potential to block the interaction of S1 protein with ACE2 and prevent SARS-CoV-2 infection. Therefore, a MagMASS assay was developed for the discovery of ligands to the S1 protein. Compared with previous MagMASS, this new assay used robotics for five-fold enhancement of throughput and sensitivity. The assay was validated using the SBP-1 peptide, which is identical to the ACE2 amino acid sequence recognized by the S1 protein, and then applied to the discovery of natural ligands from botanical extracts. Small molecule ligands to the S1 protein were discovered in extracts of the licorice species, Glycyrrhiza inflata. In particular, the licorice ligand licochalcone A was identified through dereplication and comparison with standards using HPLC with high resolution tandem mass spectrometry.

Affinity selection-mass spectrometry (AS-MS), which includes size exclusion AS-MS, pulsed ultrafiltration AS-MS, and magnetic microbead affinity selection (MagMASS), has considerable potential for facilitating the discovery of natural products with affinity for pharmacological targets. Compared with conventional high-throughput screening utilizing fluorescence or absorbance readouts (such as FRET or fluorescence polarization), AS-MS offers advantages such as compatibility with any type of ligand mixture, matrix, or assay buffer and no requirement for fluorescent tags. Uniquely. AS-MS does not suffer from interference from samples containing fluorophores or chromophores, which are common in natural products. MagMASS has also been demonstrated to be compatible with 96-well screening plate formats and automation, which is not the case for size exclusion AS-MS.

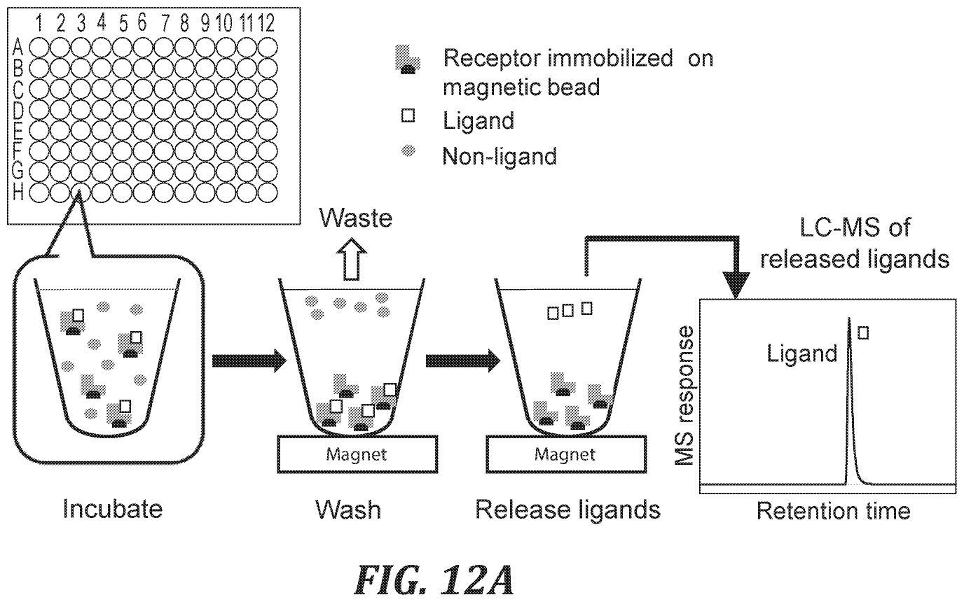

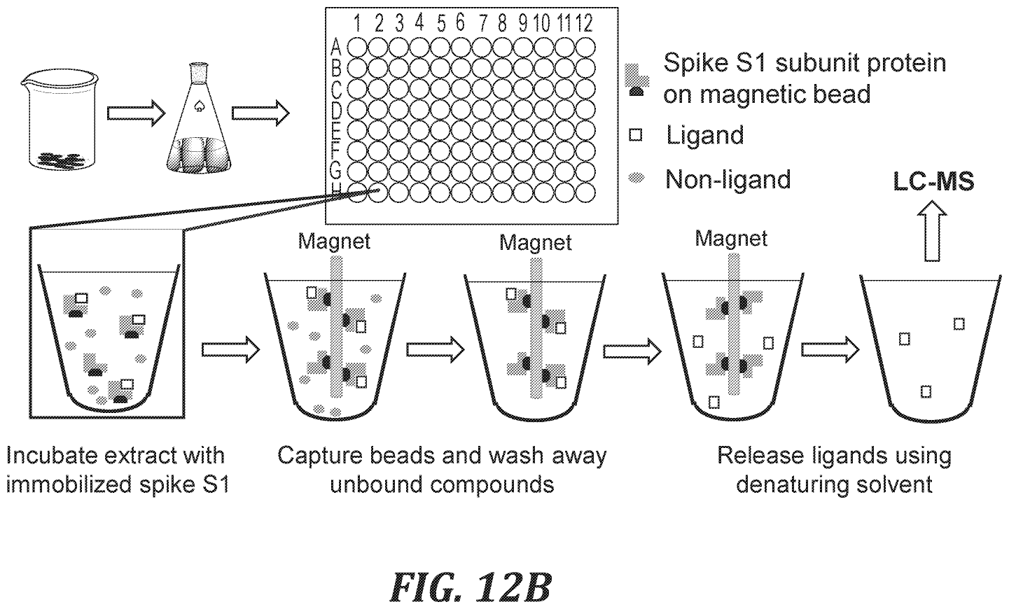

Magnetic microbeads were originally used for protein isolation and purification from complex mixtures using approaches such as batch affinity chromatography. Briefly, MagMASS utilizes a pharmacological target such as a receptor or enzyme immobilized on magnetic microbeads. Mixtures of potential ligands are incubated with the immobilized target, unbound compounds are rinsed away with binding buffer while a magnetic field retains the beads, and then ligands are released from the target using a destabilizing solution such as organic solvent for analysis using LC-MS (FIG. 12).

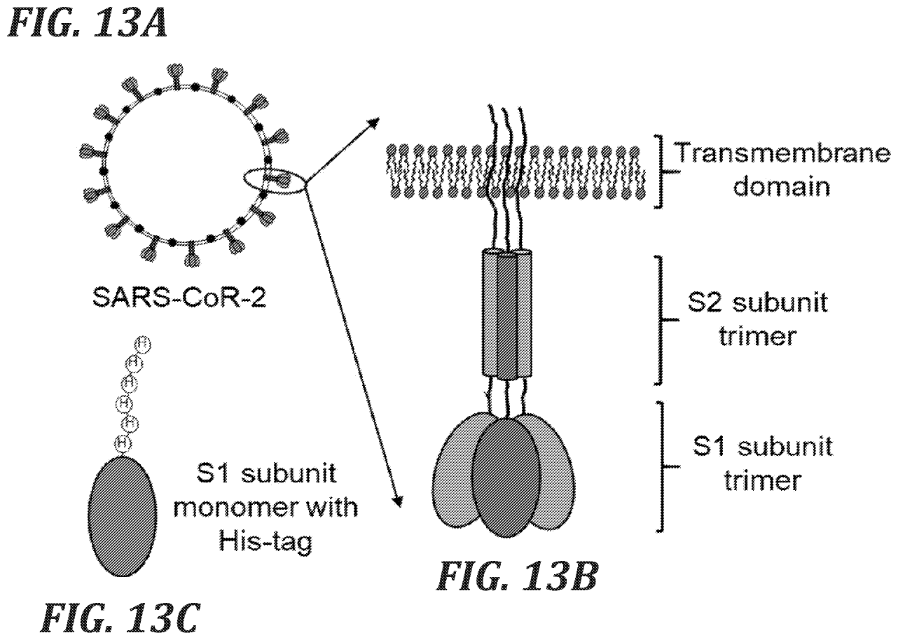

Responsible for the COVID-19 pandemic, severe acute respiratory syndrome coronavirus 2 (SARS-CoV-2) is a coronavirus, named after the characteristic crown-like spike proteins on the outer surface of the virus particles (FIG. 13). Binding of the spike protein of SARS-CoV-2 to the human cell surface receptor angiotensin converting enzyme-2 (ACE2) is the essential first step in the infection of human cells. Vaccines and antibody therapy against SARS-CoV-2 target the spike protein subunit 1 (S1; residues 331 to 524), which has high affinity for the human cell-surface protein ACE2, and prevent the virus from binding to ACE2 and infecting human cells. Small molecules with affinity to the SARS-CoV-2 S1 protein might also function as cell entry inhibitors and prevent infection or shorten the course of SARS-CoV-2 infections.

A MagMASS assay was developed to discover small molecule ligands to the SARS-CoV-2 S1 protein that might function as cell entry inhibitors and lead to the development of new therapeutic agents that prevent viral infection. The utility of the new assay was demonstrated by screening botanical extracts and discovering ligands to the S1 protein in the licorice species, Glycyrrhiza inflata. In addition, the previous MagMASS approach was enhanced 5-fold with respect to speed and sensitivity through automation using a 96-well plate magnetic particle processor.

### Materials and Reagents

Recombinant SARS-CoV-2 spike protein S1 (FIG. 13) was obtained from RayBiotech (Peachtree Corners, GA, USA). The 23-mer SBP-1 peptide was purchased from LifeTein (Hillsborough, NJ, USA). Licochalcone A, imatinib, mycophenolic acid, quinacrine, and HPLC-grade methanol and acetonitrile were purchased from Sigma Aldrich (St. Louis, MO, USA). LC-MS grade formic acid was purchased from ThermoFisher (Waltham, MA, USA). EmerTher Ni²⁺-nitrilotriacetic acid derivatized magnetic microbeads were obtained from AvanBio (Parsippany, NJ, USA). Polypropylene 2-mL conical bottom 96-well plates were purchased from Fisher Scientific (Hanover Park, Ill., USA), and ultrapure water was prepared in house using a Milli-Q water purification system (Millipore, MA, USA). Extracts of Dioscorea villosa, Glycyrrhiza glabra, Glycyrrhiza inflata, Humulus lupulus, and Trifolium pratense L. were botanically authenticated and chemically standardized as described in Rush, et al, Magnetic Microbead Affinity Selection Screening (MagMASS) of Botanical Extracts for Inhibitors of 15-Lipoxygenase. J. Nat. Prod. 2016, 79, 2898-2902, and Muchiri, et al., Affinity Selection-Mass Spectrometry for the Discovery of Pharmacologically Active Compounds from Combinatorial Libraries and Natural Products. J. Mass Spectrom. 2021, 56.

Affinity selection-mass spectrometry (MagMASS). Recombinant SARS-CoV-2 spike protein S1 subunit (˜72 kDa) containing an N-terminal His-tag (FIG. 13) was immobilized on Ni²⁺-nitrilotriacetic acid derivatized magnetic microbeads as recommended by the manufacturer. To confirm the activity of immobilized S1 protein, positive control incubations were carried out using SBP-1, a 23 amino acid peptide with the sequence of IEEQAKTFLDKFNHEAEDLFYQS (SEQ ID NO: 2), which is identical to the ACE2 α1 helix sequence recognized by the SARS-CoV-2 spike protein S1 subunit. As a negative control, denatured S1 protein, which was obtained by incubating intact S1 protein in a water bath at 95° C. for 15 min, was immobilized on identical magnetic microbeads.

Initial MagMASS assays (FIG. 12A) were carried out as described previously for the immobilized target, 15-lipoxygenase. Briefly, SBP-1 (33 nM) was incubated for 60 min with magnetic microbeads (8 μL, 50 mg/mL) containing 100 pmol of immobilized active or denatured S1 protein in 150 μL binding buffer. After washing twice with 500 μL of 30 mM ammonium acetate to remove unbound ligand while the beads were retained by a magnetic field, ligand was released from the beads using 90% methanol in water (200 μL). The samples were evaporated to dryness and reconstituted in 100 μL of water/methanol (50:50; v/v) containing 189 nM ketoconazole as internal standard for analysis using UHPLC-LC/MS. Analyses were carried out using a Shimadzu (Kyoto, Japan) Nexera UHPLC system interfaced with a Shimadzu LCMS-8050 triple quadrupole mass spectrometer. SBP-1 and ketoconazole were measured using electrospray with polarity switching followed by collision-induced dissociation with selected reaction monitoring. Reversed phase UHPLC separation was carried out using a Waters (Milford, MA, USA) Acquity UPLC BEH C₁₈ column (1.7 μm, 130 Å, 2.1 mm×50 mm) with a 3 min linear gradient from 20% to 80% acetonitrile in 0.1% aqueous formic acid at a flow rate of 0.3 mL/min. The auto-sampler temperature was 4° C., the injection volume was 10 μL, and the column oven was 40° C.

To enhance the throughput of MagMASS for botanical extract assays, a ThermoFisher KingFisher Flex Magnetic Particle Processor was used for automated processing of one 96-well plate at a time (FIG. 12B). Briefly, botanical extracts (10 μg/mL), mixtures of compounds (0.10 μM each), or individual compounds (0.10 μM) were incubated with 100 pmol of immobilized S1 protein per well. Incubations with immobilized denatured S1 protein were used to control for non-specific binding. The magnetic beads containing immobilized S1 protein in each well were captured by the magnetic probe of the KingFisher, and the contents of each well were mixed slowly for 2 out of every 5 min for 1 h. The captured magnetic beads containing immobilized S1 protein and ligands were washed twice for 1 min each in 500 μL of 30 mM ammonium acetate and washed again in 500 μL of high purity water. Ligands were eluted into 200 μL of 90% aqueous methanol (FIG. 12B). The samples were evaporated to dryness as described above and reconstituted in 100 μL of equimolar water/methanol (50:50) containing 480 nM [d4]-daidzein and 189 nM ketoconazole as internal standards.

The released ligands were analyzed using LC-MS with positive ion or negative ion electrospray on a Shimadzu LCMS-9030 Q-ToF hybrid high resolution mass spectrometer at a resolving power of 30,000 FWHM. The electrospray interface temperature was 300° C. and the voltages were 4.5 kV and −3.5 kV for positive and negative ion mode, respectively. The heat block and desolvation line temperatures were 400° C. and 250° C., respectively. Nitrogen was used as a drying gas at a flow rate of 10 L/min, as a heating gas at 10 L/min, and for nebulization at 3 L/min. Data-dependent product ion tandem mass spectrometry was used such that mass spectra and product ion tandem mass spectra were acquired every 100 ms over the scan range of m/z 100-1200 and m/z 70-1200, respectively. During data dependent acquisition, six dependent events were required at an intensity threshold of 3000, and the product ion tandem mass spectra were obtained using a collision energy of 35 V with an energy spread of 17 V.

Reversed phase HPLC separations were carried out using a Waters C₁₈ Cortecs HPLC column (2.7 μm, 2.1 mm×50 mm) with a 19 min linear gradient from 5% to 95% acetonitrile in water containing 0.1% formic acid at a flow rate of 0.3 mL/min. The column was re-equilibrated at 5% acetonitrile for 2 min between injections. The auto-sampler temperature was 4° C., the injection volume was 10 μL, and the column oven was 30° C.

Data processing. The analysis of each pair of chromatograms (intact S1 protein and denatured S1 protein negative control) was carried out using the Online XCMS (Scripps Research, La Jolla, CA USA) which is an open source metabolomics software program. To expedite data analysis, an in-house Python code was designed that enables comparison of HPLC-MS data of a botanical extract (aligned with a blank analysis) with the corresponding MagMASS HPLC-MS data (aligned with denatured receptor control data). This additional step helps eliminate false positives that may result from background noise. High resolution mass spectra were processed using Shimadzu LabSolutions V5.2 software. Natural product ligands were identified based on their elemental compositions (determined using high resolution accurate mass measurements), tandem mass spectra, and comparison with authentic standards.

Ligand binding site determination. To determine if binding occurs at the active site of the S1 protein (orthosteric) or at another site (allosteric), the binding of each ligand to the S1 protein was investigated in the presence of a molar excess of the known high affinity orthosteric ligand, SBP-1. If binding of the new ligand was reduced or eliminated through competition with SBP-1, then it would be determined to be an orthosteric ligand.

Computational modeling of hit compounds to the S1 C-terminal domain (CTD). The computational aided modeling of ligand binding to the SARS-CoV-2 spike S1 protein was carried out using AutoDock Vina (Scripps Research). The coordinates of the crystal structure of CTD of the S1 protein were downloaded from Protein Data Bank (PDB, ID number 6LZG). The ChemDraw structures of the ligands were converted to .pdb files using Pymol (Schrödinger, New York, NY). The S1-CTD was loaded into the AutoDock Vina program, the search space was defined around the known orthosteric site, and the file was converted to .pdbqt. Similarly, the ligands were individually loaded and converted to .pdbqt files. The search space x, y and z coordinates, S1-CTD, and ligand were defined on the command prompt in which the Python script developed for the AutoDock Vina was loaded and the run was initiated.

### Results

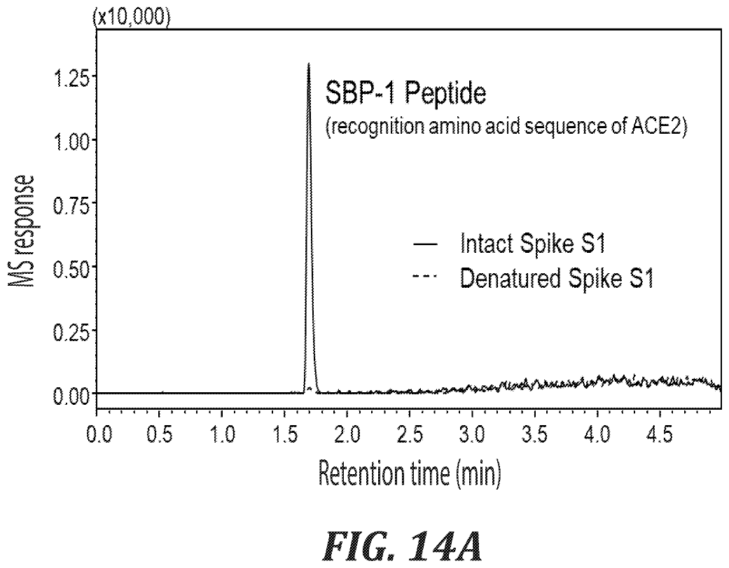

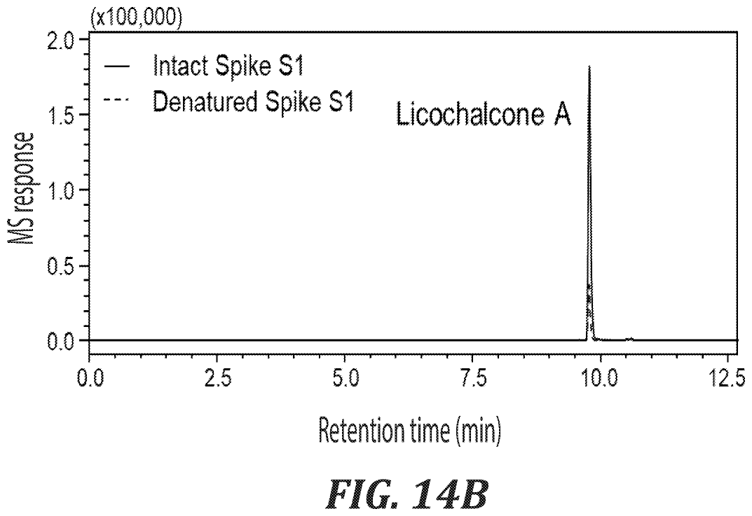

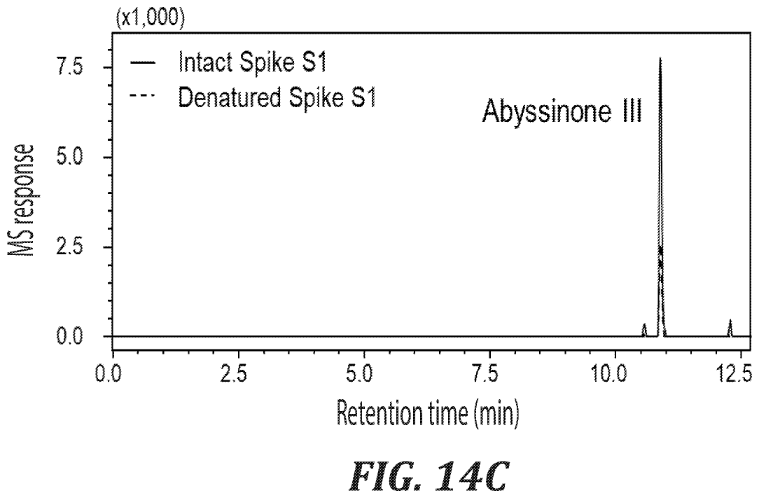

To discover natural ligands to the SARS-CoV-2 spike protein, a MagMASS assay (FIG. 12) was developed that utilizes the spike S1 protein (FIG. 13) immobilized on magnetic microbeads. To confirm that the immobilized S1 protein retained selectivity for the human cell surface protein ACE2, S1 protein immobilized on magnetic microbeads was incubated with SBP-1, which is a peptide containing the amino acid sequence of human ACE2 to which the SARS-CoV-2 spike protein recognition site (amino acid residues 331 to 524) binds. After processing using MagMASS, SBP-1 was determined to bind to the immobilized S1 protein (positive control) but not to denatured S1 subunit that had been immobilized in an identical manner (FIG. 14A).

The application of MagMASS to the screening of botanical extracts resulted in detection of two compounds from the licorice species Glycyrrhiza inflata that showed specific binding to intact spike S1 subunit (FIG. 14B). For comparison, no ligands were detected in extracts of wild yam (Dioscorea villosa), hops (Humulus lupulus), red clover (Trifolium pratense), or in the licorice species Glycyrrhiza glabra (data not shown). G. inflata ligands to the spike S1 subunit were detected at retention times of 9.8 and 10.9 min (FIGS. 14B and 14C) using high resolution positive ion electrospray mass spectrometry as protonated molecules of m/z 339.1590 and m/z 391.1907, respectively. These accurate mass measurements correspond to elemental compositions of C₂₁H₂₃O₄ (theoretical m/z 339.1596; ΔM −1.8 ppm) and C₂₅H₂₇O₄ (theoretical m/z 391.1909; AM-0.5 ppm).

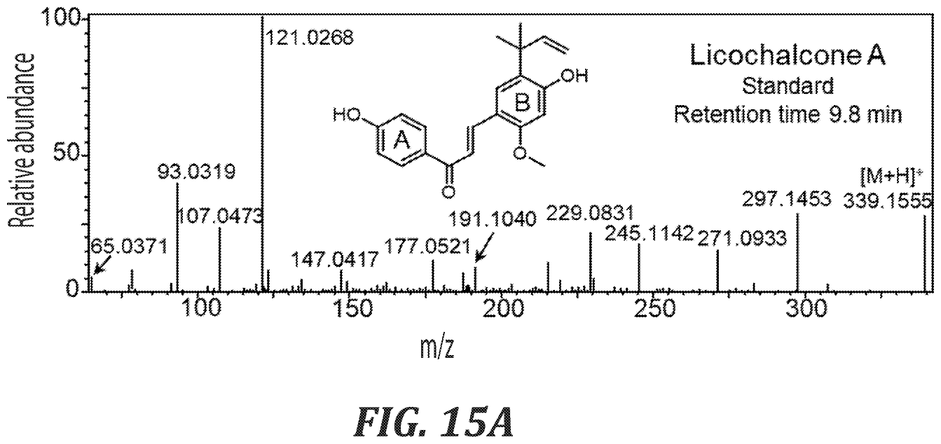

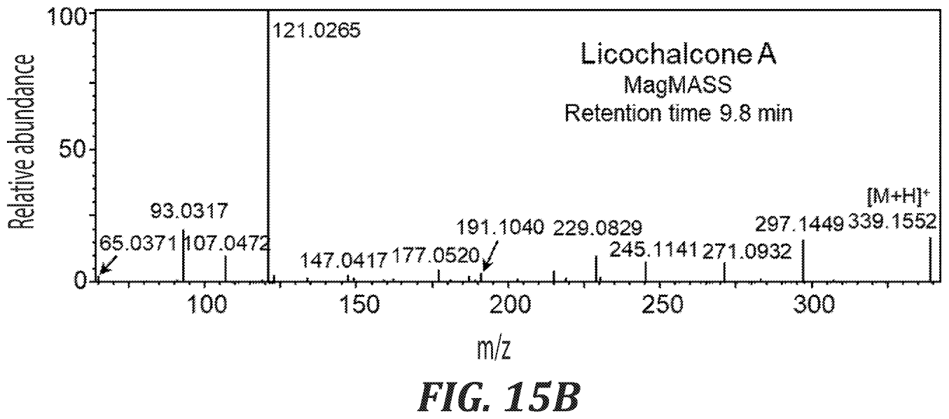

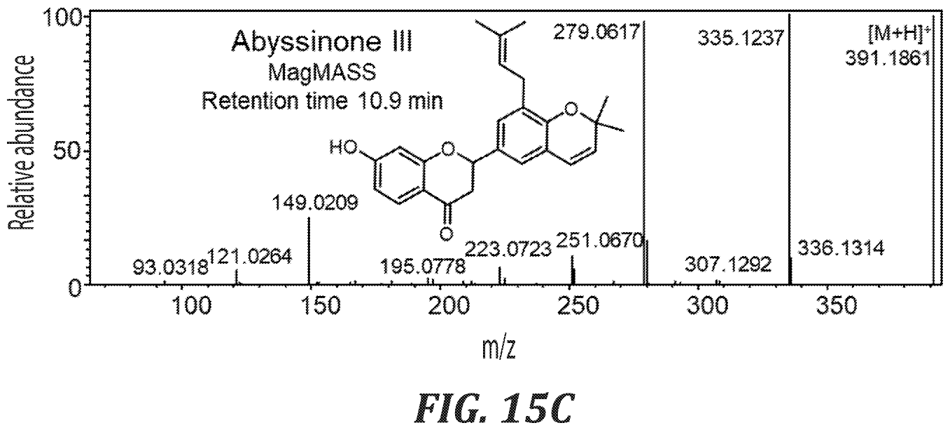

To identify these natural ligands to the SARS CoV-2 S1 protein, dereplication was carried out by searching databases such as PubChem, METLIN, and GNPS for compounds of these elemental compositions known to occur in G. inflata. Abyssinone III was a possible compound found to occur in G. inflata corresponding to an elemental composition of C₂₅H₂₆O₄ (uncharged molecule). In the absence of an authentic standard, confirmation of the identity of this ligand is ongoing. Dereplication of secondary metabolites in G. inflata with elemental compositions of C₂₁H₂₂O₄ (uncharged molecule) indicated several possibilities including licochalcone A, licochalcone C, licochalcone E, lupwigheone, and eurycarpin A. Comparison of the LC-MS retention time and high resolution tandem mass spectrum of the ligand eluting at 9.8 min with those of a licochalcone A standard confirmed that this compound was licochalcone A (FIG. 15).

Previously, imatinib, mycophenolic acid, and quinacrine were reported to block SARS-CoV-2 replication in cell culture. However, the mechanism of action of these inhibitors remains unknown. To determine if these compounds function by binding to the SARS-CoV-2 spike protein and thereby blocking cell entry, they were tested in the new MagMASS assay. Imatinib, mycophenolic acid, and quinacrine showed no binding to the S1 protein in the MagMASS assay (data not shown), which indicates that these compounds act by a different mechanism.

Furthermore, the throughput of the previous 96-well MagMASS assay was enhanced 5-fold by incorporating an automated robotic magnetic particle processor (FIG. 12). Fast separation of the ligand-receptor complexes from the unbound compounds is essential to minimize the loss of signal due to dissociation of the ligand during sample processing. This is particularly important when the dissociation of the ligand is rapid. In this MagMASS assay for the discovery of ligands to the SARS-CoV-2 S1 protein, 5-fold faster sample processing resulted in 5-fold signal enhancement. An additional feature of the MagMASS assay was the use of data-dependent acquisition of high resolution product ion tandem mass, which meant that not only elemental composition data but also structure information for ligands were acquired during a single analysis.

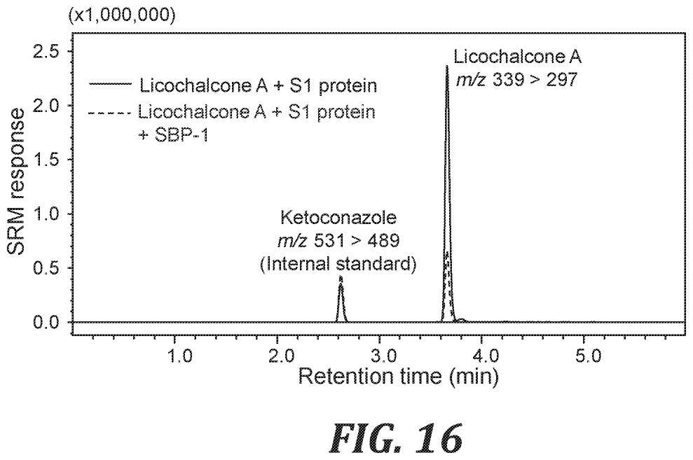

Binding of licochalcone A to the active site of the SARS-CoV-2 S1 protein was confirmed for licochalcone A by demonstrating competition for binding with SBP-1. The MagMASS peak area enrichment of licochalcone A was lowered 3-fold when SBP-1 was added to the incubation mixture containing licochalcone A and active S1 protein (FIG. 16). This observation strongly suggests that licochalcone A binds at the orthosteric site and competes with the known orthosteric ligand SBP-1.

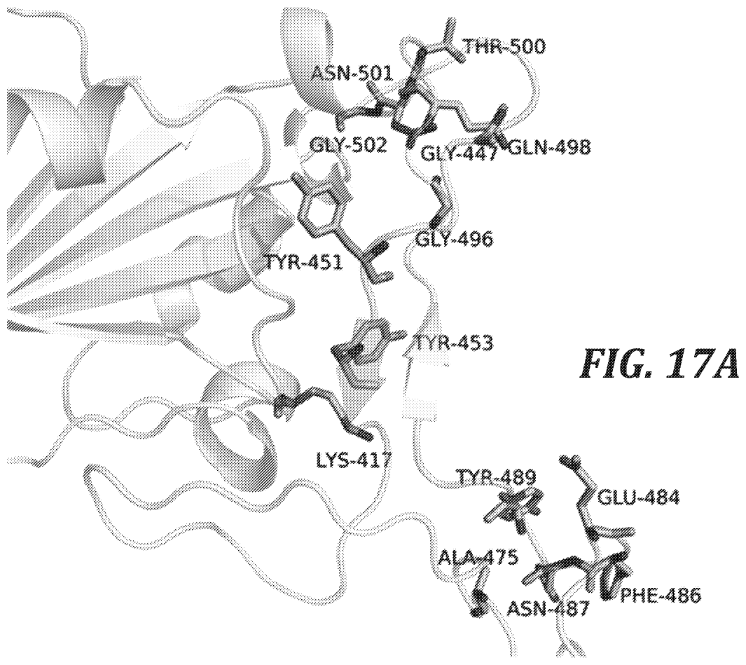

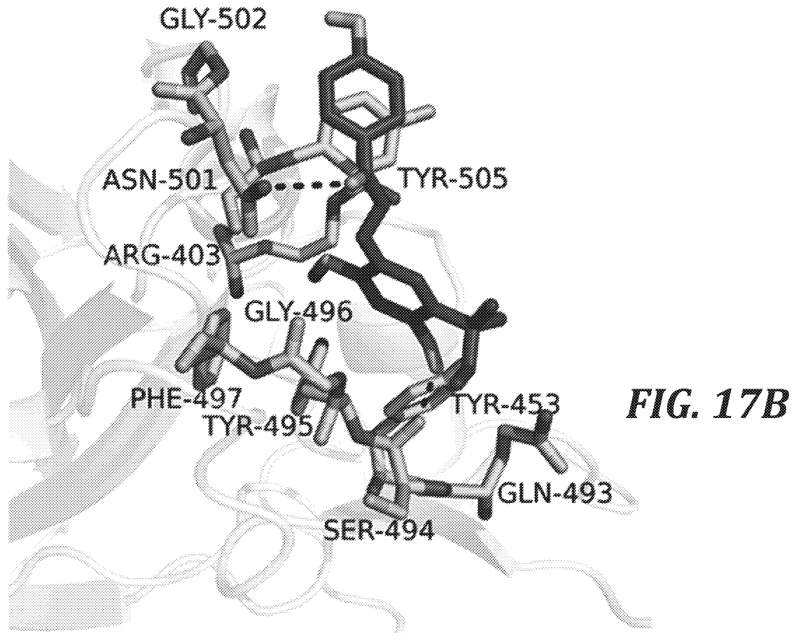

Recently, the crystal structure of the C-terminal domain (CTD) of the SARS-CoV-2 S1 protein was solved in complex with the human ACE2 receptor. The key interactions involve residues along the S1-CTD interface that contribute to a network of hydrogen-bonds and salt-bridge interactions with ACE2. These S1-CTD residues include A475, N487, E484, Y453, K417, G446, Y449, G496, Q498, T500, G502, Y489, and F486. Licochalcone A was modeled into the S1-CTD using AutoDock Vina to determine if it binds at the orthosteric site or allosterically. The search scope was based around the SARS-CoV-2 spike CTD orthosteric site (FIG. 17A) and the size of the search space in all dimensions was >25 Å to ensure the space was large enough for the ligands to rotate.

The best predicted binding mode for licochalcone A was at the orthosteric site with a free energy of binding of −6.5 kcal/mol (FIG. 17B). The key interactions within 3.5 Å involved hydrogen bonding between the hydroxyl group on the B-ring of licochalcone A and the carbonyl backbone of Ser494. Additional hydrogen bonding was predicted between the carbonyl group of licochalcone A with the side chain amino group of Asn501. The A-ring of licochalcone A was in a favorable position to form π-π stacking interaction with the aromatic ring of Tyr505. The B-ring of licochalcone A was buried in a hydrophobic pocket surrounded by Phe497, Tyr495, and Gly496.

### CONCLUSION

New applications and innovations continue to expand the utility and popularity of AS-MS. Among the suite of AS-MS approaches, MagMASS has been the most amenable to automation, and as report here is an automated version that is 5-fold faster than has been achieved previously. In addition, described herein are the application of MagMASS to the discovery of natural ligands to the SARS-CoV-2 spike protein subunit S1, which have the potential to prevent the infection of human cells by SARS-CoV-2 and be developed for use as therapeutic agents. As an application of this new assay, the discovery of licorice compounds that bind to the SARS-CoV-2 S1 protein is discussed. Like any AS-MS discovery, the activity of these licorice ligands must be determined using follow up functional assays such as cell-based virus neutralization assays using live SARS-CoV-2.

The various embodiments described above can be combined to provide further embodiments. All of the U.S. patents, U.S. patent application publications, U.S. patent applications, foreign patents, foreign patent applications and non-patent publications referred to in this specification are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

While illustrative embodiments have been illustrated and described, it will be appreciated that various changes can be made therein without departing from the spirit and scope of the invention.

1. A method for preventing infection by a β coronavirus in a subject or treating a β coronavirus-induced disease in a subject in need thereof, the method comprising administering an effective amount of a cannabinoid acid or a pharmaceutically acceptable salt, stereoisomer, or prodrug thereof, to the subject, wherein the cannabinoid acid has formula (II): 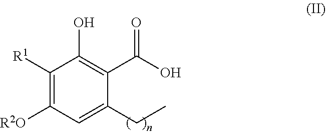
wherein
R¹ is alkenyl or alkenylcycloalkenyl;
R² is H or R¹ and R² join to form a tetrahydro- or dihydropyran ring;
n is an integer from 1 to 8,
wherein each occurrence of alkenyl and alkenylcycloalkenyl is optionally substituted with one or more substituents.

2. The method of claim 1, wherein R¹ is alkenylcycloalkenyl.

3. The method of claim 1, wherein R¹ is alkenyl.

4. The method of claim 1, wherein R¹ and R² join to form a tetrahydro- or dihydropyan ring.

5. The method of claim 1, wherein R² is H.

6. The method of claim 1, wherein the tetrahydro- or dihydropyran ring is substituted by one or more substituents selected from alkyl or alkenyl, or a combination thereof.

7. The method of claim 1, wherein n is 2.

8. The method of claim 1, wherein n is 4.

9. The method of claim 1, wherein n is 6.

10. The method of claim 1, wherein the cannabinoid acid selected from the group consisting of
cannabigerolic acid,
cannabidiolic acid,
cannabinolic acid,
cannabichromenic acid,
cannabichromevarinic acid,
cannabicyclolic acid,
cannabidivarinic acid,
cannabielsoin acid A,
cannabigerovarinic acid,
delta-8-tetrahydrocannabinolic acid,
delta-9-tetrahydrocannabinolic acid A,
delta-9-tetrahydrocannabinolic acid B,
tetrahydrocannabinolic acid,
delta-9-tetrahydrocannabivarinic acid,
tetrahydrocanabivarinic acid, and
pharmaceutically acceptable salts, stereoisomers, or prodrugs thereof.

11. The method of claim 1, wherein the cannabinoid acid is one or more of cannabigerolic acid (CBGA), tetrahydrocannabinolic acid (THCA-A), cannabidiolic acid (CBDA), or cannabinolic acid (CBNA), or a pharmaceutically acceptable salt, stereoisomer, or prodrug thereof.

12. The method of claim 1, wherein the cannabinoid acid is CBDA or a pharmaceutically acceptable salt, stereoisomer, or prodrug thereof.

13. The method of claim 1, wherein the cannabinoid acid is CBGA or a pharmaceutically acceptable salt, stereoisomer, or prodrug thereof.

14. The method of claim 1, wherein the cannabinoid acid is THCA-A or a pharmaceutically acceptable salt, stereoisomer, or prodrug thereof.

15. The method of claim 1, wherein the cannabinoid acid is CBNA or a pharmaceutically acceptable salt, stereoisomer, or prodrug thereof.

16. The method of claim 1, wherein the cannabinoid acid is a combination of CBDA, or pharmaceutically acceptable salt, stereoisomer, or prodrug thereof, and CBGA, or pharmaceutically acceptable salt, stereoisomer, or prodrug thereof.

17. The method of claim 1, wherein the cannabinoid acid is a combination of THCA-A, or pharmaceutically acceptable salt, stereoisomer, or prodrug thereof, and CBGA, or pharmaceutically acceptable salt, stereoisomer, or prodrug thereof.

18. The method of claim 1, wherein the cannabinoid acid is CBDA, or pharmaceutically acceptable salt, stereoisomer, or prodrug thereof, and wherein administering an effective amount of a cannabinoid acid or a pharmaceutically acceptable salt, stereoisomer, or prodrug thereof, further comprises administering to the subject an effective amount a compound that inhibits (efflux) transport of CBDA out of cells.

19. The method of claim 18, wherein the compound that inhibits transport of CBDA out of cells is cannabidiol (CBD) or cannabigerol (CBG).

20. A pharmaceutical composition, consisting essentially of CBDA, or pharmaceutically acceptable salt, stereoisomer, or prodrug thereof, and CBGA, or a pharmaceutically acceptable salt, stereoisomer, or prodrug thereof, and a pharmaceutically acceptable carrier.

21. A pharmaceutical composition, consisting essentially of THCA-A or pharmaceutically acceptable salt, stereoisomer, or prodrug thereof, and CBGA, or a pharmaceutically acceptable salt, stereoisomer, or prodrug thereof, and a pharmaceutically acceptable carrier.

22. A pharmaceutical composition, consisting essentially of CBDA, or pharmaceutically acceptable salt, stereoisomer, or prodrug thereof, a compound that inhibits transport of CBDA out of cells, and a pharmaceutically acceptable carrier.

23. The pharmaceutical composition of claim 22, wherein the compound that inhibits transport of CBDA out of cells is CBG or CBD.

24. The method of claim 1, wherein the cannabinoid acid or the pharmaceutically acceptable salt, stereoisomer, or prodrug thereof, is administered orally.

25. The method of claim 1, wherein the cannabinoid acid or the pharmaceutically acceptable salt, stereoisomer, or prodrug thereof, is administered topically.

26. The method of claim 1, wherein the cannabinoid acid or the pharmaceutically acceptable salt, stereoisomer, or prodrug thereof, is inhaled.


SEQUENCE LISTING (2 sequences)

SEQ ID NO: 1 - protein, 1273 aa, Betacoronavirus severe acute respiratory syndrome-related coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 23 aa, Artificial sequence
  Synthetic
IEEQAKTFLDKFNHEAEDLFYQS